(19) United States
(12) Patent Application Publication
(10) Pub. No.: US 2006/0229305 A1
(43) Pub. Date: Oct. 12, 2006

(54) Tetrahydronaphthalene derivatives, process for their production and their use as anti-inflammatory agents

(76) Inventors: Markus Berger, Berlin (DE); Stefan Baeurle, Berlin (DE); Heike Schaecke, Berlin (DE); Hartmut Rehwinkel, Berlin (DE); Norbert Schmees, Berlin (DE); Bernd Buchmann, Hohen Nuendorf (DE); Konrad Krolikiewicz, Berlin (DE); Anne Mengel, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/384,857

(22) Filed: Mar. 21, 2006

Related U.S. Application Data
(60) Provisional application No. 60/670,260, filed on Apr. 12, 2005.

(30) Foreign Application Priority Data
Mar. 22, 2005 (DE) 102005014089.0

(51) Int. Cl. A61K 31/538; A61K 31/517; A61K 31/502; A61K 31/498; A61K 31/4745; A61K 31/4706; A61K 31/416; A61K 31/4184; A61K 31/405; A61K 31/382; A61K 31/366

(52) U.S. Cl. 514/230.5; 514/266.4; 514/249; 514/313; 514/300; 514/310; 514/309; 514/312; 514/406; 514/457; 514/248; 514/419; 514/394; 514/417; 544/105; 544/237; 544/353; 546/122; 546/153; 546/159; 546/144; 548/161; 549/23; 548/305.4; 548/361.1; 548/471; 548/495; 549/303

(57) ABSTRACT

The invention relates to tetrahydronaphthalene derivatives of general formula (I) 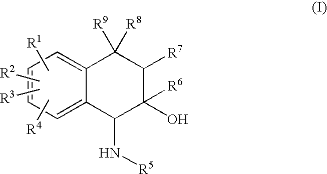 process for their production, and their use as anti-inflammatory agents.

This application claims the benefit of the filing date of U.S. Provisional Application Ser. No. 60/670,260 filed Apr. 12, 2005 which is incorporated by reference herein.

The invention relates to tetrahydronaphthalene derivatives, process for their production and their use as anti-inflammatory agents.

Open-chain, non-steroidal anti-inflammatory agents are known from the prior art WO 02/10143. In the experiment, these compounds show dissociations of action between anti-inflammatory and undesirable metabolic actions and are superior to the previously described nonsteroidal glucocorticoids or exhibit at least just as good an action.

The compounds of the prior art still have drawbacks, so that one skilled in the art is motivated in addition to seek new compounds that bind to the glucocorticoid receptors.

Compounds have now been found that have an action that is at least comparable to that of the compounds described in the prior art.

This invention relates to compounds of general formula (I), 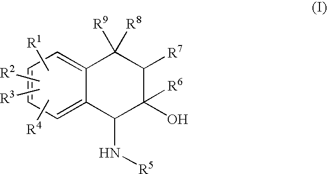
in which

R¹ and R², independently of one another, mean a hydrogen atom, a hydroxy group, a halogen atom, an optionally substituted (C₁-C₁₀)-alkyl group, an optionally substituted (C₁-C₁₀)-alkoxy group, a (C₁-C₁₀)-alkylthio group, a (C₁-C₅)-perfluoroalkyl group, a cyano group, or a nitro group, or

R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2, and the terminal atoms are linked to directly adjacent ring-carbon atoms,
or NR¹⁰OR¹¹, whereby R¹⁰ and R¹¹, independently of one another, can be hydrogen, C₁-C₅-alkyl or (CO)—C₁-C₅-alkyl,

R³ and R⁴, independently of one another, mean a hydrogen atom, a hydroxy group, a halogen atom, a cyano group, an optionally substituted (C₁-C₁₀)-alkyl group, an optionally substituted (C₁-C₁₀)-alkoxy group, a (C₁-C₁₀)-alkylthio group, or a (C₁-C₅)-perfluoroalkyl group,

R⁵ means a C₁-C₁₀-alkyl group, or a C₁-C₁₀-alkyl group that is substituted by one or more groups that are selected from hydroxy groups, halogen atoms, or (C₁-C₅)-alkoxy groups,
an optionally substituted (C₃-C₇)-cycloalkyl group,
an optionally substituted heterocyclyl group,
an optionally substituted aryl group,
a monocyclic or bicyclic heteroaryl group that optionally is substituted, independently of one another, by one or more groups selected from (C₁-C₅)-alkyl groups (which optionally can be substituted by 1-3 hydroxy or 1-3 COOR¹⁰ groups), (C₁-C₅)-alkoxy groups, hydroxy groups, halogen atoms, or 1-2 (C₁-C₃)-exoalkylidene groups or that optionally contains 1-4 nitrogen atoms and/or 1-2 oxygen atoms and/or 1-2 sulfur atoms and/or 1-2 keto groups, whereby this group can be linked via any position to the amine of the tetrahydronaphthalene system and optionally can be hydrogenated at one or more sites,

R⁶ means a (C₁-C₅)-alkyl group or an optionally partially or completely fluorinated (C₁-C₅)-alkyl group, a (C₃-C₇)-cycloalkyl group, a (C₃-C₇)-cycloalkyl(C₁-C₈)alkyl group, a (C₃-C₇)-cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkenyl group, a monocyclic or bicyclic heteroaryl group that optionally is substituted by one or more keto groups, (C₁-C₅)-alkyl groups, (C₁-C₅)-alkoxy groups, halogen atoms, or (C₁-C₃)-exoalkylidene groups and that contains one or more nitrogen atoms and/or oxygen atoms and/or sulfur atoms,
a heteroaryl(C₁-C₈)alkyl group or a heteroaryl(C₂-C₈)alkenyl group, whereby these groups can be linked via any position to the tetrahydronaphthalene system and optionally can be hydrogenated at one or more sites,

R⁷ means a halogen atom, or a (C₁-C₁₀)-alkyl group, which optionally can be substituted by OR¹⁰, SR¹⁰, N(R¹⁰R¹¹) or 1-3 halogen atoms,

R⁸ and R⁹, independently of one another, mean a hydrogen atom, a halogen atom, a (C₁-C₅)alkyl group, which can be substituted with OR¹⁰, SR¹⁰, or N(R¹⁰R¹¹), a cyano group, or, together with the carbon atom of the ring system, a (C₃-C₆)-cycloalkyl ring, or together a (C₁-C₅)-alkylidene group that optionally is substituted by hydroxy, halogen or cyano, or

R⁷ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms.

Stereoisomers of general formula (I) in which

R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkythio group, a (C₁-C₅)-perfluoroalkyl group, a cyano group, or a nitro group, or

R¹ and R² together mean a group that is selected from the groups —O—(CH₂)ₙ—O—, —O—(CH₂)ₙ—CH₂—, —O—CH═CH—, and —(CH₂)ₙ₊₂—, whereby
n=1 or 2, and the terminal atoms are linked to directly adjacent ring-carbon atoms, or NR¹⁰R¹¹, whereby R¹⁰ and R¹¹, independently of one another, can be hydrogen, C₁-C₅-alkyl or (CO)—C₁-C₅-alkyl,

R³ and R⁴, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,

R⁵ means a C₁-C₁₀-alkyl group, a C₁-C₁₀-alkyl group that is substituted by one or more groups selected from 1-3 hydroxy groups, halogen atoms, or 1-3 (C₁-C₅)-alkoxy groups, an optionally substituted phenyl group, a monocyclic or bicyclic heteroaryl group that optionally is substituted by 1-2 keto groups, 1-2 (Cₗ-C₅)-alkyl groups, 1-2 (C₁-C₅)-alkoxy groups, 1-3 hydroxy groups, 1-3 halogen atoms, or 1-2 (C₁-C₃)-exoalkylidene groups and that contains 1-4 nitrogen atoms and/or 1-2 oxygen atoms and/or 1-2 sulfur atoms and/or 1-2 keto groups, whereby these groups can be linked via any position to the amine of the tetrahydronaphthalene system and optionally can be hydrogenated at one or more sites,

R⁶ means a (C₁-C₅)-alkyl group or an optionally partially or completely fluorinated (C₁-C₅)-alkyl group, an aryl group, an aryl(C₁-C₈)alkyl group, an aryl(C₂-C₈)alkenyl group, a (C₃-C₇)cycloalkyl group, a (C₃-C₇)cycloalkyl(C₁-C₈)alkyl group, or a (C₃-C₇)cycloalkyl(C₂-C₈)alkenyl group,

R⁷ means a halogen atom, a (C₁-C₁₀)-alkyl group, which optionally can be substituted with OR¹⁰, SR¹⁰, N(R¹⁰R¹¹) or 1-3 halogen atoms,

R⁸ and R⁹, independently of one another, mean a hydrogen atom, a halogen atom, a methyl or ethyl group, which should be substituted with OR¹⁰, SR¹⁰, or NR¹⁰R¹¹, a cyano group, or, together with the carbon atom of the tetrahydronaphthalene ring, a (C₃-C₆)-cycloalkyl ring, or together a (C₁-C₅)-alkylidene group, or

R⁷ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms,

are another subject of the invention.

Stereoisomers of general formula (I) in which

R¹ and R², independently of one another, mean a hydrogen atom, a hydroxy group, a halogen atom, an optionally substituted (C₁-C₅)-alkyl group, a (C₁-C₅)-alkoxy group, or a cyano group, or

R¹ and R² together mean a group that is selected from the groups —O—(CH₂)ₙ—O—, —O—(CH₂)ₙ—CH₂—, —O—CH═CH—, and —(CH₂)ₙ₊₂—,
whereby n=1 or 2, and the terminal atoms are linked to directly adjacent ring-carbon atoms,

R³ and R⁴, independently of one another, mean a hydrogen atom, a hydroxy group, a halogen atom, an optionally substituted (C₁-C₁₀)-alkyl group, or a (C₁-C₁₀)-alkoxy group,

R⁵ means a C₁-C₁₀-alkyl group, a C₁-C₁₀-alkyl group that is substituted by one or more groups selected from 1-3 hydroxy groups or halogen atoms; a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted by one or more groups selected from 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups,
whereby these groups can be linked via any position to the amine of the tetrahydronaphthalene system and optionally can be hydrogenated at one or more sites,

R⁶ means a (C₁-C₅)-alkyl group or an optionally partially or completely fluorinated (C₁-C₅)-alkyl group,

R⁷ means a halogen atom, a methyl or ethyl group, which should be substituted with OR¹⁰, SR¹⁰, N(R¹⁰R¹¹) or 1-3 halogen atoms,

R⁸ and R⁹, independently of one another, mean a hydrogen atom, a halogen atom, a methyl or ethyl group, which should be substituted with OR¹⁰, SR¹⁰, or N(R¹⁰)₂, a cyano group, or, together with the carbon atom of the tetrahydronaphthalene ring, a (C₃-C₆)-cycloalkyl ring, or together a (C₁-C₅)-alkylidene group, or

R⁷ and R⁸ together mean an annelated five- to eight-membered, saturated or unsaturated carbocyclic or heterocyclic compound, which optionally is substituted by 1-2 keto groups, 1-2 (C₁-C₅)-alkyl groups, 1-2 (C₁-C₅)-alkoxy groups, or 1-4 halogen atoms,

are a subject of this invention.

Stereoisomers of general formula (I) in which

R¹ and R², independently of one another, mean a hydrogen atom, a hydroxy group, a halogen atom, a (C₁-C₅)-alkyl group, a (C₁-C₅)-alkoxy group, or together a group selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms,

R³ means a hydrogen atom, a hydroxy group, a halogen atom, an optionally substituted (C₁-C₁₀)-alkyl group, or a (C₁-C₁₀)-alkoxy group,

R⁴ means a hydrogen atom,

R⁵ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted by one or more groups selected from 1-2 keto groups, 1-2 (C₁-C₅)-alkyl groups, 1-2 (C₁-C₅)-alkoxy groups, 1-3 hydroxy groups, 1-3 halogen atoms, and 1-2 (C₁-C₃)-exoalkylidene groups,
whereby these groups can be linked via any position to the amine of the tetrahydronaphthalene system and optionally can be hydrogenated at one or more sites,

R⁶ means a (C₁-C₅)-alkyl group or an optionally partially or completely fluorinated (C₁-C₅)-alkyl group,

R⁷ means a halogen atom, a methyl or ethyl group,

R⁸ and R⁹, independently of one another, mean a hydrogen atom, a halogen atom, a methyl or ethyl group, or, together with the carbon atom of the tetrahydronaphthalene ring, a (C₃-C₆)-cycloalkyl ring, or together a methylene or ethylidene, or

R⁷ and R⁸ together mean an annelated five- to eight-membered, saturated or unsaturated carbocyclic compound or heterocyclic compound,

are another subject of this invention.

Stereoisomers of general formula (I) in which

R¹ and R², independently of one another, mean a hydrogen atom, a hydroxy group, a halogen atom, an optionally substituted (C₁-C₅)-alkyl group, a (C₁-C₅)-alkoxy group, or a cyano group or

R¹ and R² together mean a group that is selected from the groups —O—(CH₂)ₙ—O—, —O—(CH₂)ₙ—CH₂, —O—CH═CH—, or —(CH₂)ₙ₊₂—,
whereby n=1 or 2, and the terminal atoms are linked to directly adjacent ring-carbon atoms,

R³ and R⁴, independently of one another, mean a hydrogen atom, a hydroxy group, a halogen atom, an optionally substituted (C₁-C₅)-alkyl group, or a (C₁-C₅)-alkoxy group,

R⁵ means a C₁-C₁₀-alkyl group, a C₁-C₁₀-alkyl group that is substituted by one or more groups that are selected from 1-3 hydroxy groups or halogen atoms; a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, chromenyl, isochromenyl, chromenonyl, isochromenonyl, 1,7- or 1,8-naphthyridinyl, dihydroindolonyl, dihydroisoindolonyl, benzimidazole or indolyl group that optionally is substituted by one or more groups that are selected from 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups,
whereby these groups can be linked via any position to the amine of the tetrahydronaphthalene system and optionally can be hydrogenated at one or more sites,

R⁶ means a (C₁-C₅)-alkyl group or an optionally partially or completely fluorinated (C₁-C₅)-alkyl group,

R⁷ means a halogen atom, a methyl or ethyl group, which should be substituted with OR¹⁰, SR¹⁰, N(R¹⁰R¹¹) or 1-3 halogen atoms,

R⁸ and R⁹, independently of one another, mean a hydrogen atom, a halogen atom, a methyl or ethyl group, which should be substituted with OR¹⁰, SR¹⁰, or N(R¹⁰)₂, a cyano group, or, together with the carbon atom of the tetrahydronaphthalene ring, a (C₃-C₆)-cycloalkyl ring, or together a (C₁-C₅)-alkylidene group, or

R⁷ and R⁸ together mean an annelated five- to eight-membered, saturated or unsaturated carbocyclic compound or heterocyclic compound, which optionally is substituted by 1-2-keto groups, 1-2 (C₁-C₅)-alkyl groups, 1-2 (C₁-C₅)-alkoxy groups, or 1-4 halogen atoms,

are another subject of this invention.

Stereoisomers of general formula (I) in which

R¹ and R², independently of one another, mean a hydrogen atom, a hydroxy group, a halogen atom, a (C₁-C₅)-alkyl group, a (C₁-C₅)-alkoxy group, or together a group that is selected from the groups —O—(CH₂)ₙ—O—, —O—(CH₂)ₙ—CH₂—, —O—CH═CH═, and —(CH₂)ₙ₊₂—, whereby n=1 or 2, and the terminal atoms are linked to directly adjacent ring-carbon atoms,

R³ means a hydrogen atom, a hydroxy group, a halogen atom, an optionally substituted (C₁-C₅)-alkyl group, or a (C₁-C₅)-alkoxy group,

R⁴ means a hydrogen atom,

R⁵ means a phenyl, phthalidyl, isoindolyl, dihydroindolyl, dihydroisoindolyl, dihydroisoquinolinyl, thiophthalidyl, benzoxazinonyl, phthalazinonyl, chromenyl, isochromenyl, chromenonyl, isochromenonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted by one or more groups that are selected from 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups,
whereby these groups can be linked via any position to the amine of the tetrahydronaphthalene system and optionally can be hydrogenated at one or more sites,

R⁶ means a completely fluorinated (C₁-C₅)-alkyl group,

R⁷ means a methyl or ethyl group,

R⁸ and R⁹, independently of one another, mean a hydrogen atom, a methyl or ethyl group, or together a methylene or ethylidene group, or

R⁷ and R⁸ together mean an annelated five- to eight-membered saturated or unsaturated carbocyclic compound,

are another subject of this invention.

Stereoisomers of general formula (I) in which

R¹ and R², independently of one another, mean a hydrogen atom, a hydroxy group, a halogen atom, a (C₁-C₅)-alkyl group, or a (C₁-C₅)-alkoxy group,

R³ means a hydrogen atom or a halogen atom,

R⁴ means a hydrogen atom,

R⁵ means a monocyclic or bicyclic heteroaryl group that optionally is substituted by 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups and that contains 1-4 nitrogen atoms and/or 1-2 oxygen atoms and/or 1-2 sulfur atoms and/or 1-2 keto groups, whereby these groups can be linked via any position to the amine of the tetrahydronaphthalene system, and optionally can be hydrogenated at one or more sites,

R⁶ means a completely fluorinated (C₁-C₃)-alkyl group,

R⁷ means a methyl or ethyl group,

R⁸ and R⁹, independently of one another, mean a hydrogen atom, a methyl or ethyl group, or together a methylene or ethylidene group, or

R⁷ and R⁸ together mean an annelated five- to six-membered saturated or unsaturated carbocyclic compound,

are a preferred subject of this invention.

Stereoisomers of general formula (I) in which

R¹ and R², independently of one another, mean a hydrogen atom, a hydroxy group, a halogen atom, a (C₁-C₅)-alkyl group, or a (C₁-C₅)-alkoxy group,

R³ means a hydrogen atom, or a halogen atom,

R⁴ means a hydrogen atom,

R⁵ means a quinazolinyl, quinolonyl, isoquinolonyl, phthalazinonyl, phthalazinyl, quinolinyl, isoquinolinyl, dihydroindolyl, dihydroisoindolyl, dihydroindolonyl, dihydroisoindolonyl, isochromenyl, or isochromenonyl group that optionally is substituted, independently of one another, by one or more groups that are selected from a keto group, a (C₁-C₅)-alkyl group, or 1-2 halogen atoms,

R⁶ means a completely fluorinated (C₁-C₃)-alkyl group,

R⁷ means a methyl or ethyl group,

R⁸ and R⁹, independently of one another, mean a hydrogen atom, a methyl or ethyl group, or together a methylene or ethylidene group, or

R⁷ and R⁸ together, mean an annelated five- to six-membered saturated or unsaturated carbocyclic compound,

are a preferred subject of this invention.

Special subgroups of this invention are characterized by the definitions of claims 1 to 4 with the definition of R⁷ in which R⁷ means a (C₁-C₅)-alkyl group or a halogen atom and preferably in which R⁷ means a (C₁-C₃)-alkyl group and especially preferably in which R⁷ means a methyl or ethyl group.

### DEFINITIONS

The designation halogen atom or halogen means a fluorine, chlorine, bromine, or iodine atom. Preferred is a fluorine, chlorine or bromine atom. As a substituent for R⁵, the fluorine atom is quite especially preferred.

Alkyl groups R¹, R², R³, R⁴, R⁵, R⁶, R¹⁰ and R¹¹ can be straight-chain or branched and stand for, for example, a methyl, ethyl, n-propyl, iso-propyl, n-butyl, iso-butyl, tert-butyl or n-pentyl, 2,2-dimethylpropyl, 2-methylbutyl or 3-methylbutyl group. A C₁-C₃-alkyl group is preferred.

They can optionally be substituted by a group that is selected from 1-3 hydroxy atoms, 1-3 halogen atoms, 1-3 (C₁-C₃)alkoxy groups, and/or 1-3 COOR¹¹ groups. Hydroxy groups are preferred.

Alkyl group R⁵ has the meaning mentioned in the paragraph above, but the possible substituents are selected from the group of hydroxy, halogen, and (C₁-C₅)-alkyloxy.

The alkyl groups R⁸ and R⁹ have the meaning that is mentioned in the paragraph above, but the possible substituents are selected from the group OR¹⁰, SR¹⁰ and N(R¹⁰R¹¹), whereby R¹⁰ and R¹¹ mean hydrogen, C₁-C₅-alkyl or (CO)C₁-C₅-alkyl, and alkyl is also defined as above.

The alkoxy groups can be straight-chain or branched and stand for a methoxy, ethoxy, n-propoxy, iso-propoxy, n-butoxy, iso-butoxy, tert-butoxy- or n-pentoxy-, 2,2-dimethylpropoxy, 2-methylbutoxy or 3-methylbutoxy group. A methoxy or ethoxy group is preferred.

The alkylthio groups can be straight-chain or branched and stand for a methylthio, ethylthio, n-propylthio, iso-propylthio, n-butylthio, iso-butylthio, tert-butylthio- or n-pentylthio, 2,2-dimethylpropylthio, 2-methylbutylthio or 3-methylbutylthio group. A methylthio or ethylthio group is preferred.

For a partially or completely fluorinated alkyl group, which can be straight-chain or branched, for example, the following partially or completely fluorinated groups are considered: fluoromethyl, difluoromethyl, trifluoromethyl, fluoroethyl, 1,1-difluoroethyl, 1,2-difluoroethyl, 1,1,1-trifluoroethyl, tetrafluoroethyl, pentafluoroethyl, C₃F₇, C₃H₂F₅, C₄F₉, and C₅F₁₁. Of the latter, the trifluoromethyl group or the pentafluoroethyl group is preferred. The reagents are commercially available, or the published syntheses of the corresponding reagents are part of the prior art.

The aromatic portion of the tetrahydronaphthalene system can be substituted in 1-4 places, preferably 1-2 places. As substituents, the definitions of the claims that are cited for R¹, R², R³ and R⁴ are suitable: for R¹ and R², independently of one another, a hydrogen atom, a hydroxy group, a halogen atom, an optionally substituted (C₁-C₁₀)-alkyl group, an optionally substituted (C₁-C₁₀)-alkoxy group, a (C₁-C₁₀)-alkylthio group, a (C₁-C₅)-perfluoroalkyl group, a cyano group, or a nitro group—preferred, independently of one another, are a hydrogen atom, a hydroxy group, a halogen atom, an optionally substituted (C₁-C₅)-alkyl group, an optionally substituted (C₁-C₅)-alkoxy group, a (C₁-C₅)-alkylthio group, or a (C₁-C₃)-perfluoroalkyl group; especially preferred, independently of one another, are a hydrogen atom, a hydroxy group, a halogen atom, an optionally substituted (C₁-C₃)-alkyl group, an optionally substituted (C₁-C₃)-alkoxy group, a (C₁-C₃)-alkylthio group, or a (C₁-C₃)-perfluoroalkyl group; and quite especially preferred are substituents that are selected, independently of one another, from the group of hydroxy group, halogen atom, (C₁-C₃)-alkyl group and (C₁-C₃)-alkoxy group.

Compounds of general formula I, according to one of the claims, in which R¹ and R², independently of one another, especially preferably mean a hydrogen atom, a hydroxy group, a halogen atom, an optionally substituted (C₁-C₃)-alkyl group, an optionally substituted (C₁-C₃)-alkoxy group, a (C₁-C₃)-alkylthio group, or a (C₁-C₃)-perfluoroalkyl group and quite especially preferably mean a hydroxy group, a halogen atom, a (C₁-C₃)-alkyl group or a (C₁-C₃)alkoxy group, are a special subject of the invention.

If there is a mention of “skeleton” in the text, the tetrahydronaphthalene system is meant.

Aryl substituents R¹ and R² can form a ring by both aryl substituents together meaning a chain selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2. The terminal atoms of the above-cited groups are linked to directly adjacent aryl-ring-carbon atoms, so that an annelated ring is produced.

Substituent NR¹⁰OR¹¹ means, for example, NH₂, NH(CH₃), N(CH₃)₂, NH(C₂H₅), N(C₂H₅)₂, NH(C₃H₇), N(C₃H₇)₂, NH(C₄H₉), N(C₄H₉)₂, NH(C₅H₁₁), N(C₅H₁₁)₂, NH(CO)CH₃, NH(CO)C₂H₅, NH(CO)C₃H₇, NH(CO)C₄H₉, or NH(CO)C₅H₁₁.

The cycloalkyl group means a saturated cyclic group with 3 to 7 ring-carbon atoms that optionally is substituted by one or more groups that are selected from hydroxy groups, halogen atoms, (C₁-C₅)-alkyl groups, or (C₁-C₅)-alkoxy groups, such as, for example, cyclopropyl, methylcyclopropyl, cyclobutyl, methylcyclobutyl, cyclopentyl, methylcyclopentyl, cyclohexyl, methylcyclohexyl, cycloheptyl, or methylcycloheptyl.

The cycloalkylalkyl group means, for example, —(CH₂)-cycloalkyl, —(C₂H₄)-cycloalkyl, —(C₃H₆)-cycloalkyl, —(C₄H₈)-cycloalkyl, or —(C₅H₁₀)-cycloalkyl, whereby cycloalkyl is defined as described above.

Cycloalkylalkenyl group means, for example, —CH═CH)-cycloalkyl, —[C(CH₃)═CH]-cycloalkyl, —[CH═C(CH₃)]-cycloalkyl, —(CH═CH—CH₂)-cycloalkyl, —(CH₂—CH═CH)-cycloalkyl, —(CH═CH—CH₂—CH₂)-cycloalkyl, —(CH₂—CH═CH—CH₂)-cycloalkyl, —(CH₂—CH₂—CH═CH)-cycloalkyl, —(C(CH₃)═CH—CH₂)-cycloalkyl, or —(CH═C(CH₃)-CH₂)-cycloalkyl.

(C₁-C₃)-Exoalkylidene group is defined as a group that is bonded to the system (ring or chain) via an exo-double bond. Exomethylene is preferred.

Alkylidene group R⁸/R⁹ can have 1 to 5 carbon atoms, can be symmetrical or asymmetrical and optionally can be substituted by hydroxy, halogen or cyano groups.

The heterocyclyl group is not aromatic and can be, for example, pyrrolidine, imidazolidine, pyrazolidine, or piperidine. As substituents, hydroxy groups, halogen atoms, (C₁-C₅)-alkyl groups, and (C₁-C₅)-alkoxy groups are suitable.

Heterocyclylalkyl groups are defined as heterocyclyl groups that are bonded via a C₁-C₅-alkyl group to the skeleton, whereby the alkyl group can be straight-chain or branched.

Heterocyclylalkenyl groups are heterocyclyl groups that are bonded via an unsaturated C₂-C₅-alkyl group to the skeleton, whereby the alkenylene groups can be straight-chain or branched.

The aryl group R⁵ and R⁶ can be phenyl or naphthyl.

As substituents for the two groups, C₁-C₃-alkyl, hydroxy, C₁-C₃-alkoxy, C₁-C₃-alkylthio, halogen, cyano, COO(C₁-C₅)alkyl, COOH, N(R¹⁰R¹¹), and nitro are considered. The degree of substitution can be single or multiple and can contain several substituents that are the same or different. Mono- or di-substituted phenyl and naphthyl groups R⁵ are preferred.

The aryl groups can be partially hydrogenated and then can also carry keto, (C₁-C₃)-exoalkylidene in addition to or as an alternative to the above-cited substituents. Partially hydrogenated phenyl is defined as, e.g., cyclohexadienyl, cyclohexenyl, or cyclohexyl. A partially hydrogenated, substituted naphthalene system is, for example, 1-tetralone or 2-tetralone.

The arylalkyl group is an aryl group that is bonded via a C₁-C₈-alkyl group to a skeleton, whereby the alkyl group can be straight-chain or branched. For example, benzyl or phenethylene can be mentioned.

An arylalkenyl group is an aryl group that is bonded via a C₂-C₈-alkenyl group to a skeleton, whereby the alkenyl group can be straight-chain or branched.

The arylalkinyl group is an aryl group that is bonded via a C₂-C₈-alkinyl group to the skeleton, whereby the alkinyl group can be straight-chain or branched.

Monocyclic or bicyclic heteroaryl group R⁵ and R⁶ that can be hydrogenated at one or more sites is defined as all monocyclic or bicyclic aromatic ring systems that contain at least one heteroatom and at most seven heteroatoms. Heterocyclic systems that have 1-3 heteroatoms in the ring system and contain at least one nitrogen atom are especially preferred. Ring systems with 1-5 heteroatoms are preferred. As heteroatoms, 1-4 nitrogen atoms, 1-2 oxygen atoms and 1-2 sulfur atoms are suitable, which can occur in all sub-combinations in the ring system as long as they do not exceed the number specified for the respective heteroatom and the total maximum number of seven heteroatoms. For example, compounds of formula I in which R⁵ or R⁶ means furanyl, thiophenyl, pyrazolyl, pyrrolyl, oxazolyl, thiazolyl, imidazolyl, isoxazolyl, isothiazolyl, oxadiazolyl, triazolyl, tetrazolyl, thiadiazolyl, pyridyl, pyrimidinyl, pyridazinyl, pyrazinyl, triazinyl, azaindolizinyl, phthalidyl, thiophthalidyl, indolyl, isoindolyl, dihydroindolyl, dihydroisoindolyl, indazolyl, benzothiazolyl, indolonyl, dihydroindolonyl, isoindolonyl, dihydroisoindolonyl, benzofuranyl, benzimidazolyl, indolizinyl, isobenzofuranyl, azaindolyl, azaisoindolyl, furanopyridyl, furanopyrimidinyl, furanopyrazinyl, furanopyridazinyl, dihydrobenzofuranyl, dihydrofuranopyridyl, dihydrofuranopyrimidinyl, dihydrofuranopyrazinyl, dihydrofuranopyridazinyl, dihydrobenzofuranyl, coumarinyl, isocoumarinyl, dihydroisoquinolinyl, dihydroquinolinyl, benzoxazinonyl, phthalazinonyl, quinolinyl, isoquinolinyl, quinolonyl, isoquinolonyl, quinazolinyl, quinoxalinyl, cinnolinyl, phthalazinyl, 1,7- or 1,8-naphthyridinyl are thus part of this invention and represent a special embodiment of the invention. Heterocyclic ring systems, which contain several oxygen and sulfur atoms directly next to one another, are not subjects of the invention.

If the heteroaryl groups are partially or completely hydrogenated, compounds of formula I in which R⁵ means tetrahydropyranyl, 2H-pyranyl, 4H-pyranyl, piperidyl, tetrahydropyridyl, dihydropyridyl, 1H-pyridin-2-onyl, 1H-pyridin-4-onyl, 4-aminopyridyl, 1H-pyridin-4-ylidenaminyl, chromanyl, isochromanyl, chromenyl, isochromenyl, thiochromanyl, decahydroquinolinyl, tetrahydroquinolinyl, dihydroquinolinyl, 5,6,7,8-tetrahydro-1H-quinolin-4-onyl, decahydroisoquinolinyl, tetrahydroisoquinolinyl, dihydroisoquinolinyl, 3,4-dihydro-2H-benz[1,4]oxazinyl, 1,2-dihydro[1,3]benzoxazin-4-onyl, 3,4-dihydrobenz[1,4]oxazin-4-onyl, 3,4-dihydro-2H-benzo[1,4]thiazinyl, 4H-benzo[1,4]thiazinyl, 1,2,3,4-tetrahydroquinoxalinyl, 1H-cinnolin-4-onyl, 3H-quinazolin-4-onyl, 1H-quinazolin-4-onyl, 3,4-dihydro-1H-quinoxalin-2-onyl, 2,3-1,2,3,4-tetrahydro[1,5]naphthyridinyl, dihydro-1H-[1,5]naphthyridyl, 1H-[1,5]naphthyrid-4-onyl, 5,6,7,8-tetrahydro-1H-naphthyridin-4-onyl, 1,2-dihydropyrido[3,2-d][1,3]oxazin-4-onyl, octahydro-1H-indolyl, 2,3-dihydro-1H-indolyl, octahydro-2H-isoindolyl, 1,3-dihydro-2H-isoindolyl, 1,2-dihydroindazolyl, 1H-pyrrolo[2,3-b]pyridyl, 2,3-dihydro-1H-pyrrolo[2,3-b]pyridyl, 2,2-dihydro-1H-pyrrolo[2,3-b]pyridin-3-onyl are thus part of this invention.

Compounds of formula I in which R⁵ means a monocyclic or bicyclic heteroaryl group that optionally is substituted by one or more groups selected from (C₁-C₅)-alkyl groups (which optionally can be substituted by 1-3 hydroxy or 1-3 COOR¹⁰ groups), (C₁-C₅)-alkoxy groups, hydroxy groups, halogen atoms, or (C₁-C₃)exoalkylidene groups and that optionally contains 1-3 nitrogen atoms and/or 1-2 oxygen atoms and/or 1-2 sulfur atoms and/or 1-2 keto groups, whereby this group can be linked via any position to the amine of the tetrahydronaphthalene system and optionally can be hydrogenated at one or more sites, are preferred.

Especially preferred are compounds of formula I in which R⁵ means a monocyclic or bicyclic heteroaryl group that optionally is substituted by one or more groups that are selected from (C₁-C₅)-alkyl groups (which optionally can be substituted by 1-3 hydroxy groups or 1-3 COOR¹⁰ groups), (C₁-C₅)-alkoxy groups, hydroxy groups, halogen atoms, or (C₁-C₃)exoalkylidene groups and that optionally contains 1-3 nitrogen atoms and/or 1-2 oxygen atoms and/or 1-2 sulfur atoms and/or 1-2 keto groups, whereby this group can be linked via any position to the amine of the tetrahydronaphthalene system and optionally can be hydrogenated at one or more sites, and contains at most 3 hetereoatoms in the monocyclic ring system and at most 4 heteroatoms in the bicyclic ring system.

Compounds of general formula I in which R⁵ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted with C₁-C₅-alkyl, halogen, hydroxy, C₁-C₅-alkoxy, keto or (C₁-C₃)exoalkylidene are a preferred subject of the invention.

Compounds of general formula I in which R⁵ means a phenyl, phthalidyl, isoindolyl, dihydroindolyl, dihydroisoindolyl, dihydroisoquinolinyl, thiophthalidyl, benzoxazinonyl, phthalazinonyl, coumarinyl, isocoumarinyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted with C₁-C₅-alkyl, halogen, hydroxy, C₁-C₅-alkoxy, keto or (C₁-C₃)exoalkylidene are a preferred subject of the invention.

Compounds of general formula I in which R⁵ means a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, chromenyl, isochromenyl, chromenonyl, isochromenonyl, quinazolinyl, quinoxalinyl, cinnolinyl, phthalazinyl, 1,7- or 1,8-naphthyridinyl, dihydroindolonyl, dihydroisoindolyl, benzimidazole or indolyl group that optionally is substituted, independently of one another, with one or more C₁-C₅-alkyl, halogen, hydroxy, C₁-C₅-alkoxy, keto or (C₁-C₃)exoalkylidene groups are a preferred subject of the invention.

Compounds of general formula I in which R⁵means a phenyl, phthalidyl, isoindolyl, dihydroindolyl, dihydroisoindolyl, dihydroisoquinolinyl, thiophthalidyl, benzoxazinonyl, phthalazinonyl, coumarinyl, isocoumarinyl, chromenyl, isochromenyl, chromenonyl, isochromenonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted, independently of one another, with one or more C₁-C₅-alkyl, halogen, hydroxy, C₁-C₅-alkoxy, keto or (C₁-C₃)exoalkylidene groups are a preferred subject of the invention.

Compounds of general formula I in which R⁵ means a phenyl or naphthyl, phthalidyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted with C₁-C₅-alkyl, halogen, hydroxy, or C₁-C₅-alkoxy are a preferred subject.

Compounds of general formula I, according to one of the claims, in which R⁵ means a quinazolinyl, quinolonyl, isoquinolonyl, phthalazinonyl, phthalazinyl, quinolinyl, isoquinolinyl, dihydroindolyl, dihydroisoindolyl, or isochromenonyl group that optionally is substituted with C₁-C₃-alkyl, halogen, hydroxy, or C₁-C₃-alkoxy are quite especially preferred. In particular, there are compounds of general formula I, according to one of the claims, in which R⁵ means a quinazolinyl, quinolonyl, isoquinolonyl, phthalazinonyl, quinolinyl, dihydroindolyl, dihydroisoindolyl, or isochromenonyl group that optionally is substituted with C₁-C₃-alkyl, halogen, hydroxy or C₁-C₃-alkoxy.

If this is a heteroarylalkyl group, it is understood to include an optionally also partially hydrogenated heteroaryl group as described above, which is bonded to the skeleton via a C₁-C₈-alkyl group, which can be straight-chain or branched.

If this is a heteroarylalkenyl group, it is understood to include an optionally also partially hydrogenated heteroaryl group as described above, which is bonded to the skeleton via a (C₂-C₈)-alkenyl group, which can be straight-chain or branched.

If R⁷ and R⁸ form a five- to eight-membered carbocyclic compound or heterocyclic compound (also substituted), a tricyclic system is then present.

As heteroatoms, nitrogen, oxygen or sulfur are suitable. As substituents, all radicals that are defined for R¹ are suitable.

If R⁷ and R⁸ form a carbocyclic compound, then a five- to six-membered carbocyclic compound is preferred.

Compounds of general formula I in which R⁶ means a (C₁-C₅)-alkyl group or an optionally partially or completely fluorinated (C₁-C₅)-alkyl group, a (C₃-C₇)-cycloalkyl group, a (C₃-C₇)-cycloalkyl(C₁-C₈)alkyl group, a (C₃-C₇)-cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, or an aryl(C₂-C₈)alkenyl group are another subject of the invention.

Compounds of general formula I in which R⁶ means a (C₁-C₅)-alkyl group or an optionally partially or completely fluorinated (C₁-C₅)-alkyl group, an aryl group, an aryl(C₁-C₈)alkyl group, an aryl(C₂-C₈)alkenyl group, a (C₃-C₇)-cycloalkyl group, a (C₃-C₇)-cycloalkyl(C₁-C₈)alkyl group, or a (C₃-C₇)-cycloalkyl(C₂-C₈)alkenyl group are a subject of the invention.

Compounds of general formula I in which R⁶ represents a (C₁-C₃)-alkyl group or an optionally partially or completely fluorinated (C₁-C₃)-alkyl group are another subject of the invention. The completely fluorinated alkyl groups are especially preferred. The CF₃ group is quite especially preferred.

Compounds of formula I in which R⁶ means a C₁-C₁₀-alkyl group, which optionally can be substituted by 1-3 hydroxy groups, halogen atoms, an optionally substituted phenyl group, a monocyclic or bicyclic heteroaryl group that optionally is substituted by 1-2 keto groups, 1-2 (C₁-C₅)-alkyl groups, 1-2 (C₁-C₅)-alkoxy groups, 1-3 halogen atoms, or 1-2 (C₁-C₃)exoalkylidene groups or that contains 1-4 nitrogen atoms and/or 1-2 oxygen atoms and/or 1-2 sulfur atoms, whereby these groups can be linked via any position to the nitrogen atom and optionally can be hydrogenated at one or more sites, are another subject of the invention.

The compounds of general formula I according to the invention can be present as stereoisomers because of the presence of asymmetry centers. All possible stereoisomers (e.g.: RRRR, RRRS, RRSR, RSRR, SRRR, RSRS, RRSS, RSSR, SRRS, SSRR, SRSR, RSSS, SRSS, SSRS, SSSR, SSSS), both as racemates and in enantiomer-pure form, and both as pure diastereomers and as diastereomer mixtures, are subjects of this invention.

The compounds according to the invention can also be present in the form of salts with physiologically compatible anions, for example in the form of hydrochloride, sulfate, nitrate, phosphate, pivalate, maleate, fumarate, tartrate, benzoate, mesylate, citrate or succinate.

Esters or ethers or amides of the compounds of general formula I or other compounds that metabolize in the organism to form compounds of general formula I are also subjects of this invention.

The compounds according to the invention are produced either

a) by styrenes of general formula (II), produced according to methods that are known in the prior art, being converted by an optionally enantioselectively conducted En reaction with chiral Lewis acids into the compounds of general formula (III). As chiral Lewis acids, the following can be used: (R)- or (S)-SEGPHOS-PdCl₂ (Mikami et al. Tetrah. Asymm. 2004, 15, 3885-89), (R)- or (S)-BINOL-Ti(OiPr)₂ (Ding et al. Tetrah. Lett. 2004, 45, 2009-12), (R)- or (S)-Cu ^{t}BuBOX,), (R)- or (S)-Cu ⁱPrBOX, (R)- or (S)-Cu PhBOX, (R)- or (S)-Cu AdaBOX (Evans et al. J. Am. Chem. Soc. 2000, 122, 7936-43), (R)- or (S)-Ph-pybox Sc(OTf)₃ (Evans et al. J. Am. Chem. Soc. 2005, 127, 8006-7), (R)- or (S)-ⁱPr-pybox Yb(OTf)₃, (R)- or (S)-^{t}Bu-pybox Yb(OTf)₃, (R)- or (S)-Ph-pybox Yb(OTf)₃ (Qian et al. Tetrah. Asymm. 2000, 11, 2347-57). Imine (IV) is produced by reduction and amination according to methods that are known to one skilled in the art, 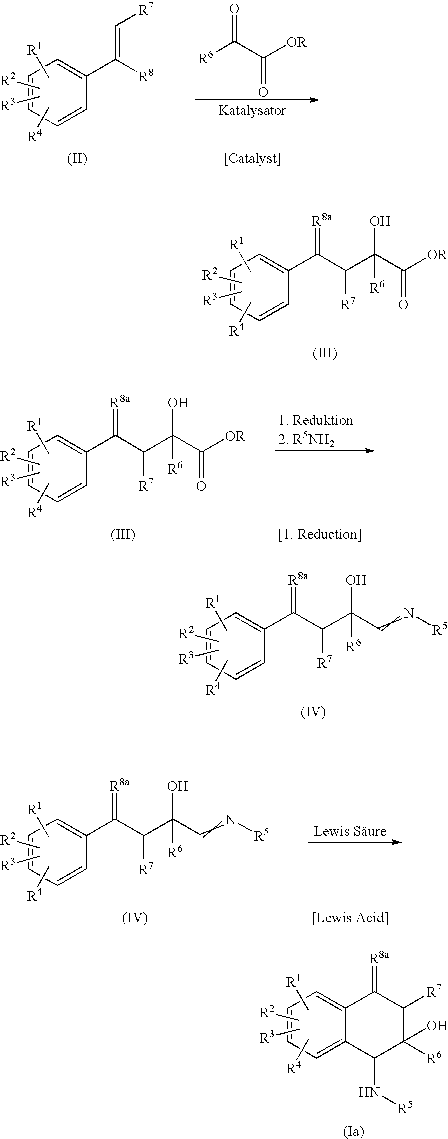
which then is cyclized either without additional reagent in a solvent, preferably chlorinated hydrocarbons, such as, e.g., methylene chloride or dichloroethane or concentrated organic acids, preferably glacial acetic acid, or by adding inorganic or organic acids or Lewis acids under temperatures in the range of −7° C. to +80° C. (preferably in the range of −30° C. to +80° C.) to form the compounds of general formula (Ia) (for R⁸+R⁹═R^{8a}=alkylidene), or

b) by the compounds of general formula (III), produced according to method a), being converted by hydrogenation into the compound of formula (V) (for R⁹═H) or by methods of cyclopropanation that are known to one skilled in the art (J. Am. Chem. Soc. 80 (1958) pp. 5323-5324, J. Org. Chem. 50 (1985), pp. 4412-4414) into compounds of formula (V) (for R⁸—R⁹═CH₂—CH₂) or by hydrohalogenation (J. Org. Chem. 53.(1988), pp. 1475-1481) into compounds of formula (V) (for R⁹═halogen).

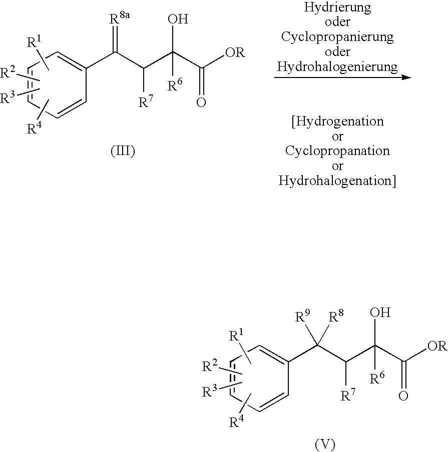

By reduction and amination, imine (VI) is produced analogously to a), which then is cyclized either without additional reagent in a solvent, preferably 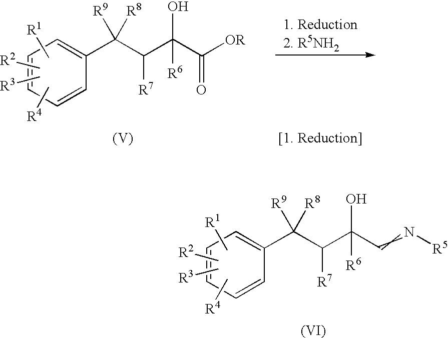
chlorinated hydrocarbons, such as, e.g., methylene chloride or dichloroethane or concentrated organic acids, preferably glacial acetic acid, or by adding inorganic or organic acids or Lewis acids under temperatures in the range of −70° C. to +80° C. (preferably in the range of −30° C. to +80° C.) to form the compounds of the general formula for compound (I).

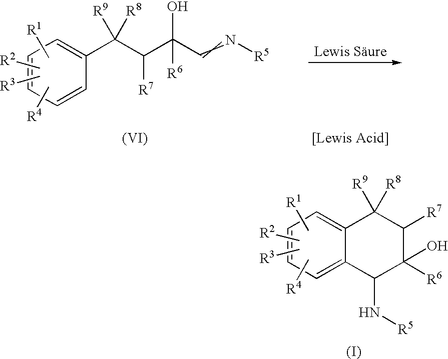

The binding of the substances to the glucocorticoid receptor (GR) and other steroid-hormone receptors (mineral corticoid receptor (MR), progesterone receptor (PR) and androgen receptor (AR)) is examined with the aid of recombinantly produced receptors. Cytosol preparations of Sf9 cells, which had been infected with recombinant baculoviruses that code for the GR, are used for the binding studies. In comparison to the reference substance [³H]-dexamethasone, the substances show a high affinity to the GR. IC₅₀(GR)=36 nM and IC₅₀(PR)>1 μM were measured for the compound from Example 3.

The GR-mediated inhibition of the transcription of cytokines, adhesion molecules, enzymes and other pro-inflammatory factors is considered to be an essential, molecular mechanism for the anti-inflammatory action of glucocorticoids. This inhibition is produced by an interaction of the GR with other transcription factors, e.g., AP-1 and NF-kappa-B (for a survey, see Cato, A. C. B., and Wade, E., BioEssays 18, 371-378, 1996).

The compounds of general formula I according to the invention inhibit the secretion of cytokine IL-8 into the human monocyte cell line THP-1 that is triggered by lipopolysaccharide (LPS). The concentration of the cytokines was determined in the supernatant by means of commercially available ELISA kits. The compound of Example 3 showed an inhibition IC₅₀(IL8)=130 nM at an 80% efficiency relative to [³H]-dexamethasone as a standard.

The anti-inflammatory action of the compounds of general formula I was tested in the animal experiment by tests in the croton oil-induced inflammation in rats and mice (J. Exp. Med. (1995), 182, 99-108). To this end, croton oil in ethanolic solution was applied topically to the animals' ears. The test substances were also applied topically or systemically at the same time or two hours before the croton oil. After 16-24 hours, the ear weight was measured as a yardstick for inflammatory edema, the peroxidase activity as a yardstick for the invasions of granulocytes, and the elastase activity as a yardstick for the invasion of neutrophilic granulocytes. In this test, the compounds of general formula I inhibit the three above-mentioned inflammation parameters both after topical administration and after systemic administration.

One of the most frequent undesirable actions of a glucocorticoid therapy is the so-called “steroid diabetes” [cf., Hatz, H. J., Glucocorticoide: Immunologische Grundlagen, Pharmakologie und Therapierichtlinien, [Glucocorticoids: Immunological Principles, Pharmacology and Therapy Guidelines], Wissenschaftliche Verlagsgesellschaft mbH, Stuttgart, 1998]. The reason for this is the stimulation of gluconeogenesis in the liver by induction of the enzymes responsible in this respect and by free amino acids, which are produced from the degradation of proteins (catabolic action of glucocorticoids). A key enzyme of the catabolic metabolism in the liver is tyrosinamino transferase (TAT). The activity of this enzyme can be determined from liver homogenates by photometry and represents a good measurement of the undesirable metabolic actions of glucocorticoids. To measure the TAT induction, the animals are sacrificed 8 hours after the test substances are administered, the livers are removed, and the TAT activity is measured in the homogenate. In this test, at doses in which they have an anti-inflammatory action, the compounds of general formula I induce little or no tyrosinamino transferase.

Because of their anti-inflammatory action, and, in addition, anti-allergic, immunosuppressive and antiproliferative action, the compounds of general formula I according to the invention can be used as medications for treatment or prophylaxis of the following pathologic conditions in mammals and humans: In this case, the term “DISEASE” stands for the following indications:
(i) Lung diseases that are accompanied by inflammatory, allergic and/or proliferative processes:
   Chronic, obstructive lung diseases of any origin, primarily bronchial asthma
   Bronchitis of different origins
   All forms of restrictive lung diseases, primarily allergic alveolitis,
   All forms of pulmonary edema, primarily toxic pulmonary edema
   Sarcoidoses and granulomatoses, especially Boeck's disease
(ii) Rheumatic diseases/autoimmune diseases/joint diseases that are accompanied by inflammatory, allergic and/or proliferative processes:
   All forms of rheumatic diseases, especially rheumatoid arthritis, acute rheumatic fever, polymyalgia rheumatica
   Reactive arthritis
   Inflammatory soft-tissue diseases of other origins
   Arthritic symptoms in the case of degenerative joint diseases (arthroses)
   Traumatic arthritides
   Collagenoses of any origin, e.g., systemic lupus erythematodes, sclerodermia, polymyositis, dermatomyositis, Sjögren's syndrome, Still's syndrome, Felty's syndrome
(iii) Allergies that are accompanied by inflammatory and/or proliferative processes:
   All forms of allergic reactions, e.g., Quincke's edema, hay fever, insect bites, allergic reactions to pharmaceutical agents, blood derivatives, contrast media, etc., anaphylactic shock, urticaria, contact dermatitis
(iv) Vascular inflammations (vasculitides)
   Panarteritis nodosa, temporal arteritis, erythema nodosum
(v) Dermatological diseases that are accompanied by inflammatory, allergic and/or proliferative processes:
   Atopic dermatitis (primarily in children)
   Psoriasis
   Pityriasis rubra pilaris
   Erythematous diseases, triggered by different noxae, e.g., radiation, chemicals, burns, etc.
   Bullous dermatoses
   Diseases of the lichenoid group
   Pruritis (e.g., of allergic origin)
   Seborrheal eczema
   Rosacea
   Pemphigus vulgaris
   Erythema exudativum multiforme
   Balanitis
   Vulvitis
   Hair loss such as alopecia areata
   Cutaneous T-cell lymphoma
(vi) Kidney diseases that are accompanied by inflammatory, allergic and/or proliferative processes:
   Nephrotic syndrome
   All nephritides
(vii) Liver diseases that are accompanied by inflammatory, allergic and/or proliferative processes:
   Acute liver cell decomposition
   Acute hepatitis of different origins, e.g., viral, toxic, pharmaceutical agent-induced
   Chronic aggressive hepatitis and/or chronic intermittent hepatitis
(viii) Gastrointestinal diseases that are accompanied by inflammatory, allergic and/or proliferative processes:
   Regional enteritis (Crohn's disease)
   Colitis ulcerosa
   Gastritis
   Reflux esophagitis
   Ulcerative colitis of other origins, e.g., native sprue
(ix) Proctologic diseases that are accompanied by inflammatory, allergic and/or proliferative processes:
   Anal eczema
   Fissures
   Hemorrhoids
   Idiopathic proctitis
(x) Eye diseases that are accompanied by inflammatory, allergic and/or proliferative processes:
   Allergic keratitis, uveitis, iritis
   Conjunctivitis
   Blepharitis
   Optic neuritis
   Chorioiditis
   Sympathetic ophthalmia
(xi) Diseases of the ear-nose-throat area that are accompanied by inflammatory, allergic and/or proliferative processes:
   Allergic rhinitis, hay fever
   Otitis externa, e.g., caused by contact dermatitis, infection, etc.
   Otitis media
(xii) Neurological diseases that are accompanied by inflammatory, allergic and/or proliferative processes:
   Cerebral edema, primarily tumor-induced cerebral edema
   Multiple sclerosis
   Acute encephalomyelitis
   Meningitis
   Various forms of convulsions, e.g., infantile nodding spasms
(xiii) Blood diseases that are accompanied by inflammatory, allergic and/or proliferative processes:
   Acquired hemolytic anemia
   Idiopathic thrombocytopenia
(xiv) Tumor diseases that are accompanied by inflammatory, allergic and/or proliferative processes:
   Acute lymphatic leukemia
   Malignant lymphoma
   Lymphogranulomatoses
   Lymphosarcoma
   Extensive metastases, mainly in breast, bronchial and prostate cancers
(xv) Endocrine diseases that are accompanied by inflammatory, allergic and/or proliferative processes:
   Endocrine orbitopathy
   Thyreotoxic crisis
   De Quervain's thyroiditis
   Hashimoto's thyroiditis
   Basedow's disease
(xvi) Organ and tissue transplants, graft-versus-host disease
(xvii) Severe shock conditions, e.g., anaphylactic shock, systemic inflammatory response syndrome (SIRS)
(xviii) Substitution therapy in:
   Innate primary suprarenal insufficiency, e.g., congenital adrenogenital syndrome
   Acquired primary suprarenal insufficiency, e.g., Addison's disease, autoimmune adrenalitis, meta-infective tumors, metastases, etc.
   Innate secondary suprarenal insufficiency, e.g., congenital hypopituitarism
   Acquired secondary suprarenal insufficiency, e.g., meta-infective tumors, etc.
(xix) Vomiting that is accompanied by inflammatory, allergic and/or proliferative processes:
   e.g., in combination with a 5-HT3 antagonist in cytostatic-agent-induced vomiting
(xx) Pains of inflammatory origins, e.g., lumbago.

Moreover, the compounds of general formula I according to the invention can be used for treatment and prophylaxis of additional pathologic conditions that are not mentioned above, for which synthetic glucocorticoids are now used (see in this respect Hatz, H. J., Glucocorticoide: Immunologische Grundlagen, Pharmakologie und Therapierichtlinien, Wissenschaftliche Verlagsgesellschaft mbH, Stuttgart, 1998).

All previously mentioned indications (i) to (xx) are described in more detail in Hatz, H. J., Glucocorticoide: Immunologische Grundlagen, Pharmakologie und Therapierichtlinien, Wissenschaftliche Verlagsgesellschaft mbH, Stuttgart, 1998.

For the therapeutic actions in the above-mentioned pathologic conditions, the suitable dose varies and depends on, for example, the active strength of the compound of general formula I, the host, the type of administration, and the type and severity of the conditions that are to be treated, as well as the use as a prophylactic agent or therapeutic agent.

The invention also relates to combination therapies or combined compositions, in which a glucocorticoid receptor (GR) agonist of formula (I) or a pharmaceutically acceptable salt thereof or a pharmaceutical composition that contains a GR agonist of formula (I) or a pharmaceutically acceptable salt thereof is administered either simultaneously (optionally in the same composition) or in succession together with one or more pharmaceutical agents for treating one of the above-mentioned pathologic conditions. For example, for treatment of rheumatoid arthritis, osteoarthritis, COPD (chronic obstructive lung disease), asthma or allergic rhinitis, a GR agonist of this invention can be combined with one or more pharmaceutical agents for treating such a condition. When such a combination is administered by inhalation, the pharmaceutical agent that is to be combined can be selected from the following list:
A PDE4 inhibitor including an inhibitor of the PDE4D isoform;
A selective β.sub2.adrenoceptor agonist, such as, for example, metaproterenol, isoproterenol, isoprenaline, albuterol, salbutamol, formoterol, salmeterol, terbutaline, orciprenaline, bitolterol mesylate, pirbuterol or indacaterol;
A muscarine receptor antagonist (for example, an M1, M2 or M3 antagonist, such as, for example, a more selective M3 antagonist), such as, for example, ipratropium bromide, tiotropium bromide, oxitropium bromide, pirenzepine or telenzepine;
A modulator of the chemokine receptor function (such as, for example, a CCR1 receptor antagonist); or
An inhibitor of the p38 kinase function.

For another subject of this invention, such a combination with a GR agonist of formula (D or a pharmaceutically acceptable salt thereof is used to treat COPD, asthma or allergic rhinitis and can be administered by inhalation or orally in combination with xanthine (such as, for example, aminophylline or theophylline), which also can be administered by inhalation or orally.

The invention also relates to the use of the claimed compounds/stereoisomers for the production of a pharmaceutical agent.

In addition, the invention provides:
(i) The use of one of the compounds of general formula I according to the invention or mixture thereof for the production of a medication for treating a DISEASE;
(ii) A process for treating a DISEASE, said process comprises an administration of an amount of the compound according to the invention, whereby the amount suppresses the disease and whereby the amount of compound is given to a patient who requires such a medication;
(iii) A pharmaceutical composition for treating a DISEASE, said treatment comprises one of the compounds according to the invention or mixture thereof and at least one pharmaceutical adjuvant and/or vehicle.

The compounds of general formula I according to the invention are especially suitable for the production of a medication for treatment or prophylaxis of inflammatory diseases.

In particular, the use of the compounds according to the invention for the production of a medication for treatment of a disease that is cited under i), ii), iii), iv), v) and x) is a subject of this invention.

In general, satisfactory results can be expected in animals when the daily doses comprise a range of 1 μg to 100,000 μg of the compound according to the invention per kg of body weight. In the case of larger mammals, for example the human, a recommended daily dose lies in the range of 1 pg to 100,000 μg per kg of body weight. Preferred is a dose of 10 to 30,000 μg per kg of body weight, and more preferred is a dose of 10 to 10,000 μg per kg of body weight. For example, this dose is suitably administered several times daily. For treating acute shock (e.g., anaphylactic shock), individual doses can be given that are significantly above the above-mentioned doses.

The formulation of the pharmaceutical preparations based on the new compounds is carried out in a way that is known in the art by the active ingredient being processed with the vehicles, fillers, substances that influence decomposition, binding agents, moisturizers, lubricants, absorbents, diluents, flavoring correctives, coloring agents, etc., that are commonly used in galenicals and being converted into the desired form of administration. In this case, reference is made to Remington's Pharmaceutical Science, 15^{th} Edition, Mack Publishing Company, East Pennsylvania (1980).

For oral administration, especially tablets, coated tablets, capsules, pills, powders, granulates, lozenges, suspensions, emulsions or solutions are suitable.

For parenteral administration, injection and infusion preparations are possible.

For intra-articular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local treatment.

For pulmonary administration of the new compounds, the latter can be used in the form of aerosols and inhalants.

For local application to eyes, outer ear channels, middle ears, nasal cavities, and paranasal sinuses, the new compounds can be used as drops, ointments and tinctures in corresponding pharmaceutical preparations.

For topical application, formulations in gels, ointments, fatty ointments, creams, pastes, powders, milk and tinctures are possible. The dosage of the compounds of general formula I should be 0.01%-20% in these preparations to achieve a sufficient pharmacological action.

The invention also comprises the compounds of general formula I according to the invention as therapeutic active ingredients. In addition, the compounds of general formula I according to the invention are part of the invention as therapeutic active ingredients together with pharmaceutically compatible and acceptable adjuvants and vehicles.

The invention also comprises a pharmaceutical composition that contains one of the pharmaceutically active compounds according to the invention or mixtures thereof or a pharmaceutically compatible salt thereof and pharmaceutically compatible adjuvants and vehicles.

Experiments

### EXAMPLE 1

### 2-Fluoro-5-[(8-fluoro-2-methylquinazolin-5-yl)amino]-5,6,7,8-tetrahydro-7,8-dimethyl-6-(trifluoromethyl)naphthalene-1,6-diol

### 5-Amino-8-fluoro-2-methylquinazoline

A solution of 2.4 g (18.6 mmol) of 2,5-difluoroaniline in 11 ml of water and 1.6 ml of concentrated hydrochloric acid (37%), which is 50° C. and which was previously stirred for 1 hour at this temperature, is added to a solution of 3.35 g (20.25 mmol) of chloral hydrate and 21.27 g (149.7 mmol) of sodium sulfate in 72 ml of water. It is stirred for another 30 minutes at room temperature, and after 4.09 g (58.9 mmol) of hydroxylammonium chloride in 19 ml of water is added, it is heated over 45 minutes to 125° C. and kept at this temperature for 5 minutes. After cooling and after another hour, the precipitated light-brown precipitate is filtered off, washed with water and dried. 3.0 g (15.0 mmol) of the hydroxylimine is obtained as an intermediate product, which is dissolved in portions in 15 ml of concentrated sulfuric acid at 60° C. After the addition is completed, it is heated for 2 hours to 80° C. and for 4 hours to 90° C. It is allowed to cool off, and the solution is poured onto 100 g of ice. It is extracted with ethyl acetate, the organic phase is washed with water, dried on sodium sulfate and concentrated by evaporation. After chromatography on silica gel with hexane-ethyl acetate (0-45%), 1.2 g (7.1 mmol) of 4,7-difluoroisatin is obtained. Over 10 minutes, 1.8 ml of a 30% hydrogen peroxide solution is added in drops to isatin in 30 ml of a 1 molar sodium hydroxide solution. After 2 hours of stirring at room temperature, it is cooled to 0° C., and 5 ml of a 4 molar hydrochloric acid is added and diluted with 50 ml of water. It is extracted with ethyl acetate, dried on sodium sulfate, concentrated by evaporation, and 1.27 g of the 3,6-difluoroanthranilic acid, which is reacted without further purification, is thus obtained quantitatively.

The 3,6-difluoroanthranilic acid is heated in 8 ml of acetic acid anhydride for 45 minutes to 100° C. After cooling, the acetic acid that is produced and excess acetic acid anhydride are removed azeotropically with toluene in a vacuum. The residue is mixed with 40 ml of a 25% ammonia solution while being cooled with ice, and it is stirred for 72 hours. It is diluted with water and acidified with acetic acid. It is extracted with ethyl acetate, the organic phase is washed with water, dried on sodium sulfate and concentrated by evaporation. The thus obtained 1.03 g (5.25 mmol) of 5,8-difluoro-2-methyl-3H-quinazolin-4-one and 6 g of phosphorus-pentachloride are heated in 20 ml of phosphoryl chloride over 12 hours to 125° C. After cooling, it is poured into saturated NaHCO₃ solution and extracted with ethyl acetate. The organic phase is dried, and the solvent is removed. 1.7 g of 4-chloro-5,8-difluoro-2-methylquinazoline, which is dissolved in 60 ml of ethyl acetate and 5 ml of triethylamine, is obtained quantitatively. 600 mg of palladium on carbon is added, and it is shaken for 2 hours (480 ml of hydrogen absorption) under a hydrogen atmosphere at normal pressure. Catalyst is removed from the solution by means of filtration over Celite, whereby it is rewashed with 100 ml of ethanol and concentrated by evaporation. After chromatography on silica gel with hexane-ethyl acetate-ethanol (0-40%), 550 mg of 5,8-difluoro-2-methylquinazoline is obtained. 890 mg (13.7 mmol) of sodium azide is added to 240 mg (1.3 mmol) of 5,8-difluoro-2-methylquinazoline, 300 mg (1.13 mmol) of 18-crown-6 in 10 ml of DMF, and the mixture is heated over 8 hours to 125° C. The solvent is removed in a vacuum, and it is chromatographed on silica gel with ethyl acetate, and 52 mg of product is obtained.

¹H-NMR (300 MHz, CDCl₃); δ=2.92 (s, 3H), 4.31 (br., 2H), 6.67 (dd, 1H), 7.38 (dd, 1H), 9.37 (s, 1H).

### 4-(3-Fluoro-2-methoxyphenyl)-2-hydroxy-3-methyl-2-(trifluoromethyl)pentanal

19.6 ml of propionic acid chloride is added in drops to 20 ml (213 mmol) of 2-fluorophenol in 24 ml of pyridine at 0° C. It is stirred for 1.5 hours at room temperature, and then 200 ml of a 1 M hydrochloric acid is added. It is extracted several times with dichloromethane, washed with water, dried with sodium sulfate and concentrated by evaporation in a vacuum. 38.4 g of propionic acid-(2-fluorophenyl)ester is obtained as a crude product, which is added in drops in 30 ml of dichlorobenzene to 29 g of aluminium trichloride. The reaction mixture is stirred for 18 hours at 100° C. After cooling, it is diluted with dichloromethane, carefully poured into cold 4 M hydrochloric acid and vigorously stirred for 10 more minutes. It is extracted several times with dichloromethane, washed with water, dried with sodium sulfate and concentrated by evaporation in a vacuum. After chromatographic purification on silica gel (hexane/ethyl acetate 00-40%), 16.7 g (99 mmol) of 1-(3-fluoro-2-hydroxyphenyl)-propan-1-one is obtained. 25.1 g (89 mmol) of potassium carbonate and 10.9 ml (70 mmol) of methyl iodide are added to 16.7 g of 1-(3-fluoro-2-hydroxyphenyl)-propan-1-one in 150 ml of acetone. It is heated for 6 hours to 70° C., and then the solvent is distilled off to about 80%. The residue is added to water, and it is extracted several times with diethyl ether. It is washed with water and dried on sodium sulfate. The solvent is removed in a vacuum, and 16.8 g (92 mmol) of 1-(3-fluoro-2-hydroxyphenyl)-propan-1-one is obtained.

¹H-NMR (CDCl₃): δ=1.18 (t, 3H), 2.97 (q, 2H), 3.99 (s, 3H), 7.02 (ddd, 1H), 7.20 (dd, 1H), 7.34 (d, 1H).

58 g (890 mmol) of zinc powder and 1.21 g of lead dichloride are suspended in 600 ml of tetrahydrofuran, and 54 ml of dibromomethane is slowly added in drops. It is stirred for 30 more minutes, and the mixture is cooled to 0° C. 103 ml (103 mmol) of a 1 M titanium tetrachloride solution in dichloromethane is added such that the internal temperature does not exceed 10° C. It is stirred for 30 more minutes, and 18.8 g (103 mmol) of 1-(3-fluoro-2-hydroxyphenyl)-propan-1-one in 85 ml of tetrahydrofuran is added at 0° C. After 1 hour, it is diluted with diethyl ether, and the reaction mixture is carefully poured into a cold 4 M hydrochloric acid, whereby the temperature increases up to about 35° C. It is extracted several times with diethyl ether, washed with water and dried on sodium sulfate. The solvent is removed in a vacuum, and after chromatographic purification on silica gel (hexane/ethyl acetate 0%-5%), 4.8 g (26.6 mmol) of 1-fluoro-2-methoxy-3-(1-methylpropenyl)-benzene is obtained as an E/Z mixture.

6.8 g (40 mmol) of ethyltrifluoropyruvate and 1.2 g (2 mmol) of ytterbium triflate are added to 3.6 g (20 mmol) of 1-fluoro-2-methoxy-3-(1-methylpropenyl)-benzene in 4 ml of dichloroethane, and the mixture is heated over 20 hours to 140° C. After cooling, it is chromatographed directly on silica gel (hexane/ethyl acetate 20%), and 4.1 g (11.7 mmol) of 4-(3-fluoro-2-methoxyphenyl)-2-hydroxy-3-methyl-2-(trifluoromethyl)pent-4-enoic acid ethyl ester is obtained as a stereoisomer mixture. 0.9 g (2.5 mmol) of 4-(3-fluoro-2-methoxyphenyl)-2-hydroxy-3-methyl-2-(trifluoromethyl)pent-4-enoic acid ethyl ester is dissolved in 20 ml of methanol and 0.5 ml of acetic acid, and 50 mg of palladium on carbon (10%) is added. The reaction mixture is shaken under hydrogen atmosphere for 5 hours. Then, it is filtered through Celite, rewashed with dichloromethane and methanol, and the solvent is removed in a vacuum. After two cycles of co-evaporation with toluene, 850 mg of crude 4-(3-fluoro-2-methoxyphenyl)-2-hydroxy-3-methyl-2-(trifluoromethyl)pentanoic acid ethyl ester is obtained, which is cooled in 30 ml of diethyl ether to −5° C. and is added in solid form in portions to 182 mg (4.8 mmol) of lithium aluminum hydride. It is stirred for 1 hour at 0° C. and poured into saturated ammonium chloride solution. It is extracted several times with ethyl acetate, washed with saturated sodium chloride solution and dried on sodium sulfate. After chromatographic purification on silica gel (hexane/ethyl acetate 20%), 290 mg (0.9 mmol) of 4-(3-fluoro-2-methoxyphenyl)-2-hydroxy-3-methyl-2-(trifluoromethyl)pentanal is obtained as a stereoisomer mixture.

¹H-NMR (CDCl₃): δ=0.80-0.90 (d, 3H), 1.32-1.58 (d, 3H), 2.40-2.65 (dq, 1H), 3.10-3.70 (dq, 1H), 3.90 (s, 3H), 6.85-7.10 (m, 3H), 9.00-9.70 (s, 1H).

280 mg (0.90 mmol) of 4-(3-fluoro-2-methoxyphenyl)-2-hydroxy-3-methyl-2-(trifluoromethyl)pentanal, 100 mg (0.56 mmol) of 5-amino-8-fluoro-2-methylquinazoline and 0.4 ml of titanium tetraethylate are stirred in 8 ml of toluene for 2 hours at 100° C. After cooling, it is poured into water, and vigorous stirring is continued. The suspension is filtered through Celite, and it is rewashed thoroughly with ethyl acetate. The phases of the filtrate are separated, and it is extracted again with ethyl acetate. It is dried on sodium sulfate, the solvent is removed in a vacuum, and 360 mg of 4-(3-fluoro-2-methoxyphenyl)1-[(8-fluoro-2-methylquinazolin-5-yl)imino]-3-methyl-2-(trifluoromethyl)-pentan-2-ol is obtained as a crude product. 7.7 ml (7.7 mmol) of a 1 M boron tribromide solution is added in drops to 360 mg (0.6 mmol) of imine in 20 ml of CH₂Cl₂ at −30° C. The cooling bath is removed, and after 30 minutes, the batch is mixed with saturated NaHCO₃ solution, the phases are separated, the aqueous phase is extracted with CH₂Cl₂, the combined organic phases are dried (Na₂SO₄) and concentrated by evaporation in a vacuum. Column chromatography on silica gel (hexane/ethyl acetate/methanol 43:43:12%) and subsequent crystallization from chloroform provide 40 mg (0.09 mmol) of product as a racemic mixture of the main diastereomer.

¹H-NMR (300 MHz, CD₃OD): δ=1.20 (d, 3H), 1.49 (d, 3H), 2.47 (dq, 1H), 2.83 (s, 3H), 3.58 (dq, 1H), 5.17 (s, 1H); 6.75 (dd, 1H), 6.88 (dd, 1H), 6.90 (dd, 1H), 7.58 (dd, 1H), 9.60 (s, 1H).

### EXAMPLE 2

### 5-{[6-Fluoro-3,4-dimethyl-2,5-dihydroxy-2-trifluoromethyl-1,2,3,4-tetrahydronaphthalen-1-yl]amino}guinolin-2(1H)-one

### 5-Aminoquinolin-2(1H)-on

4.5 g of 5-nitroquinolin-2(1H)-one (Chem. Pharm. Bull. (1981), 29, pp. 651-56) is hydrogenated in 200 ml of ethyl acetate and 500 ml of methanol in the presence of 450 mg of palladium on activated carbon as a catalyst under normal pressure with hydrogen until the reaction is completed. The catalyst is removed by filtration through diatomaceous earth, and the reaction solution is concentrated by evaporation in a vacuum. 3.8 g of the title compound is obtained as a yellow solid.

¹H-NMR (DMSO): δ=5.85 (bs, 2H), 6.27 (d, 1H), 6.33 (d, 1H), 6.43 (d, 1H), 7.10 (t, 1H), 8.07 (d, 1H), 11.39 (br, 1H)

290 mg (0.94 mmol) of 4-(3-fluoro-2-methoxyphenyl)-2-hydroxy-3-methyl-2-(trifluoromethyl)pentanal, 150 mg (0.94 mmol) of 5-aminoquinolin-2(1H)-one and 0.4 ml of titanium tetraethylate are stirred in 8 ml of toluene for 2 hours at 100° C. After cooling off, it is poured into water, and vigorous stirring is continued. The suspension is filtered through Celite and rewashed thoroughly with ethyl acetate. The phases of the filtrate are separated, and it is extracted again with ethyl acetate. It is dried on sodium sulfate, the solvent is removed in a vacuum, and 480 mg of 5-{[3-fluoro-2-methoxyphenyl)-2-hydroxy-3-methyl-2-(trifluoromethyl)pentylidene]amino}quinolin-2(1H)-one is obtained as a crude product. 9.5 ml (9.5 mmol) of 1 M boron tribromide solution is added in drops at −35° C. over 10 minutes to 480 mg of imine in 19 ml of CH₂Cl₂. It is allowed to heat for 2 hours to −20° C., and the batch is poured into cold saturated NaHCO₃ solution. It is rinsed with ethyl acetate, the phases are separated, the aqueous phase is extracted with ethyl acetate, the combined organic phases are dried (Na₂SO₄) and concentrated by evaporation in a vacuum. Column chromatography on silica gel (hexane/ethyl acetate/methanol 43:43:12%) and subsequent HPLC (Gemini C18 5μ, water+0.1% TFA/acetonitrile 38−50%) provide 30 mg of product as a racemic mixture of the main diastereomer.

¹H-NMR (300 MHz, CD₃OD): δ=1.14 (d, 3H), 1.44 (d, 3H), 2.39 (dq, 1H), 3.52 (dq, 1H), 5.07 (s, 1H), 6.46 (d, 1H), 6.63 (d, 1H), 6.67 (d, 1H), 6.70 (dd, 1H), 7.36 (t, 1H), 8.15 (d, 1H).

### EXAMPLE 2A/2B

### 5-{[6-Fluoro-3,4-dimethyl-2,5-dihydroxy-2-trifluoromethyl-1,2,3,4-tetrahydronaphthalen-1-yl]amino}-quinolin-2(1H)-one is cleaved by means of preparative chiral HPLC (Chiracel OD 5μ) into the Enantiomer-Pure Compounds

Enantiomer 1: analytic HPLC: Rₜ=8.4 minutes (Chiralcel OD 5μ, 250×4.6 mm,
   hexane/ethanol 5=>50% in 20 minutes, 1 ml/minute of flow)
Enantiomer 2: analytic HPLC: Rₜ=15.1 minutes (Chiralcel OD 5μ, 250×4.6 mm,
   hexane/ethanol 5=>50% in 20 minutes, 1 ml/minute of flow)

### EXAMPLE 3

### 2-Fluoro-5-[(7-fluoro-2-methylquinazolin-5-yl)amino]-5,6,7,8-tetrahydro-7,8-dimethyl-6-(trifluoromethyl)naphthalene-1,6-diol

### 5-Amino-7-fluoro-2-methyquinazoline

17 g (70.5 mmol) of 3,6-difluoro-2-N-pivaloylaminobenzaldehyde (L. Florvall, I. Fagervall, L.-G. Larsson, S. B. Ross, Eur. J. Med. Chem. 34 (1999) 137-151), 9.2 g of acetamidine hydrochloride, 13.4 g of potassium carbonate and 10.4 g of molecular sieve (4A) are combined in 70 ml of butyronitrile. While being stirred vigorously it is heated for 17 hours to 145° C., and the solvent is removed in a vacuum. After the residue is chromatographed on silica gel with hexane/ethyl acetate (0-70%), 4.5 g of 7-fluoro-5-N-pivaloylamino-2-methyquinazoline is obtained.

1 g (3.82 mmol) of 7-fluoro-5-N-pivaloylamino-2-methyquinazoline is dissolved in 74 ml of toluene and cooled to −70° C. 9.5 ml (11.4 mmol) of a 1.2 M disobutylaluminum hydride solution in toluene is added in drops over 30 minutes. The reaction mixture is allowed to heat to −40° C., and it is stirred for 4 hours at −40° C. Water is slowly added, and it is stirred for 30 minutes at room temperature until a precipitate is formed, which is removed by means of filtration through Celite. The phases are separated, washed with saturated sodium chloride solution and dried on sodium sulfate. After chromatography on silica gel with hexane-ethyl acetate (0-100%), 64 mg of the product is obtained.

¹H-NMR (300 MHz, CDCl₃); δ=2.83 (s, 3H), 4.67 (br., 2H), 6.50 (dd, 1H), 6.93 (dd, 1H), 9.23 (s, 1H).

261 mg (0.85 mmol) of 4-(3-fluoro-2-methoxyphenyl)-2-hydroxy-3-methyl-2-(trifluoromethyl)pentanal, 150 mg (0.85 mmol) of 5-amino-7-fluoro-2-methylquinazoline and 0.5 ml of titanium tetraethylate are stirred in 8 ml of toluene for 2 hours at 100° C. After cooling, it is poured into water, and vigorous stirring is continued. The suspension is filtered through Celite, and it is rewashed thoroughly with ethyl acetate. The phases of the filtrate are separated, and it is extracted again with ethyl acetate. It is dried on sodium sulfate, the solvent is removed in a vacuum, and 4-(3-fluoro-2-methoxyphenyl)1-[(7-fluoro-2-methylquinazolin-5-yl)imino]-3-methyl-2-(trifluoromethyl)-pentan-2-ol is obtained as a crude product. 8.5 ml (8.5. mmol) of a 1 M boron tribromide solution is added in drops to imine in 17 ml of CH₂Cl₂ at −35° C. over 10 minutes. It is allowed to heat to −20° C. over 2 hours, and the batch is then poured into cold, saturated NaHCO₃ solution. It is rinsed with ethyl acetate, the phases are separated, the aqueous phase is extracted with ethyl acetate, the combined organic phases are dried (Na₂SO₄) and concentrated by evaporation in a vacuum. Column chromatography on silica gel (hexane/ethyl acetate/methanol 43:43:12%) and subsequent chromatography on an amino phase provide 55 mg of product as a racemic mixture of the main diastereomer.

¹H-NMR (300 MHz, CD₃OD): δ=1.17 (d, 3H), 1.46 (d, 3H), 2.44 (dq, 1H), 2.73 (s, 3H), 3.54 (dq, 1H), 5.15 (s, 1H), 6.69 (dd, 1H), 6.73 (d, 1H), 6.74 (d, 1H), 6.87 (dd, 1H), 9.47 (s, 1H).

### EXAMPLE 3A/3B

### 2-Fluoro-5-[(7-fluoro-2-methylquinazolin-5-yl)amino]-5,6,7,8-tetrahydro-7,8-dimethyl-6-(trifluoromethyl)naphthalene-1,6-diol is Cleaved by means of Preparative Chiral HPLC (Chiracel OD 5μ) into the Enantiomer-Pure Compounds

Enantiomer 1: analytical HPLC: Rₜ=7.9 minutes (Chiralcel OD 5μ, 250×4.6 mm,
   hexane/ethanol 5=>20% in 30 minutes, 1 ml/min of flow)
Enantiomer 2: analytical HPLC: Rₜ=13.6 minutes (Chiralcel OD 5μ, 250×4.6 mm,
   hexane/ethanol 5=>20% in 20 minutes, 1 ml/minute of flow)
      In a Similar Way, There can be Produced:

### EXAMPLE 4

### 5-{[6-Fluoro-2,5-dihydroxy-3,4-dimethyl-2-trifluoromethyl-1,2,3,4-tetrahydronaphthalen-1-yl]amino}-2-methylphthalazin-1-one

### 5-Amino-2-methyl-phthalazin-1-one

### 3-Bromo-4-nitro-phthalide

5.37 g of 4-nitrophthalide (Tetrahedron Lett. (2001), 42, pp. 1647-50), 8.04 g of N-bromosuccinimide and 196 mg of benzoyl peroxide are heated in 80 ml of benzotrifluoride under reflux and with exposure to light until the reaction is completed. It is added to water, extracted with dichloromethane, washed several times with water, dried, and the solvent is removed in a vacuum. 7.24 g of 3-bromo-4-nitro-phthalide is obtained as a solid.

¹H-NMR (300 MHz, CDCl₃), δ=7.26 (s, 1H), 7.88 (t, 1H), 8.3 (d, 1H), 8.56 (d, 1H)

### 5-Nitro-phthalazin-1-one

18.25 g of hydrazine sulfate and 14.88 g of sodium carbonate are stirred in 300 ml of DMF at 100° C. for 1 hour. Then, 7.24 g of 3-bromo-4-nitro-phthalide in 100 ml of DMF is added, and it is stirred for another 4 hours at 100° C. It is added to water, extracted several times with ethyl acetate, and the organic phase is washed with water and brine. It is dried, and the solvent is removed in a vacuum. After recrystallization from ethyl acetate, 2.35 g of 5-nitro-phthalazin-1-one is obtained as a solid.

¹H-NMR (300 MHz, DMSO-d₆), δ=8.05 (t, 1H), 8.57-8.66 (m, 2H), 8.73 (s, 1H), 13.13 (bs, 1H)

### 2-Methyl-5-nitro-phthalazin-1-one

1.6 g of 5-nitro-phthalazin-1-one and 2.31 g of potassium carbonate are stirred for 10 minutes at room temperature in 60 ml of DMF. 1.1 ml of methyl iodide is added, and it is stirred overnight. It is added to water, extracted several times with ethyl acetate, and the organic phase is washed with water and brine. It is dried, and the solvent is removed in a vacuum. 1.57 g of 2-methyl-5-nitro-phthalazin-1-one is obtained as a yellow solid.

¹H-NMR (300 MHz, DMSO-d₆), δ=3.73 (s, 3H), 8.05 (t, 1H), 8.62 (d, 2H), 8.75 (s, 1H)

### 5-Amino-2-methyl-phthalazin-1-one

1.57 g of 2-methyl-5-nitro-phthalazin-1-one and 130 mg of palladium on activated carbon are suspended in 45 ml of ethyl acetate and hydrogenated with hydrogen under normal pressure. It is filtered through diatomaceous earth, and the solvent is removed in a vacuum. 1.26 g of 5-amino-2-methyl-phthalazin-1-one is obtained as a yellow solid.

¹H-NMR (300 MHz, CDCl₃), =3.81 (s, 3H), 7.0 (d, 1H), 7.5 (t, 1H), 7.8 (d, 1H), 8.16 (s, 1H)

The desired product can be produced analogously to Example 1 from 5-amino-2-methyl-phthalazin-1-one and 4-(3-fluoro-2-methoxyphenyl)-2-hydroxy-3-methyl-2-(trifluoromethyl)pentanal.

### EXAMPLE 5

### 2-Fluoro-5,6,7,8-tetrahydro-7,8-dimethyl-5-[(2-methylquinazolin-5-yl)amino]-6-(trifluoromethyl)naphthalene-1,6-diol

### 5-Amino-2-methylquinazoline

12.7 g (62 mmol) of 2-methyl-5-nitro-3H-quinazolin-4-one (M. T. Bogert, V. J. Chambers J. Org Chem. 1905, 649-658) and 37.5 g of phosphorus pentachloride are refluxed in 75 ml of phosphoryl chloride over 20 hours. After cooling, it is poured into saturated NaHCO₃ solution and extracted with ethyl acetate. The organic phase is dried, and the solvent is removed. 14 g of 4-chloro-2-methyl-5-nitroquinazoline, of which 4.5 g (20.2 mmol) is dissolved in 225 ml of ethyl acetate and 22.5 ml of triethylamine, is obtained. 2 g of palladium on carbon is added, and it is stirred for 4 hours, while being cooled with ice, under hydrogen atmosphere at normal pressure. Catalyst is removed from the solution by means of filtration over Celite, and it is rewashed with 200 ml of ethanol and concentrated by evaporation. After chromatography on silica gel with ethyl acetate-ethanol (0-10%), 530 mg of the product is obtained.

¹H-NMR (300 MHz, CDCl₃); δ=2.87 (s, 3H), 4.52 (br., 2H), 6.77 (d, 1H), 7.33 (d, 1H), 7.65 (t, 1H), 9.40 (s, 1H).

The desired product can be produced analogously to Example 1 from 5-amino-2-methylquinazoline and-4-(3-fluoro-2-methoxyphenyl)-2-hydroxy-3-methyl-2-(trifluoromethyl)pentanal.

### EXAMPLE 6

### 5-[(7,8-Difluoro-2-methylquinazolin-5-yl)amino]-2-fluoro-5,6,7,8-tetrahydro-7,8-dimethyl-6-(trifluoromethyl)naphthalene-1,6-diol

### 5-Amino-7,8-difluoro-2-methylquinazoline

156 ml (391 mmol) of a 2.5 M butyllithium solution in hexane is added in drops to 41.7 g (180 mmol) of 2,2-dimethyl-N-(3,4,5-trifluorophenyl)-propionamide in 385 ml of THF at −70° C. It is allowed to stir for one hour, and then 38.6 ml of DMF in 90 ml of THF is added in drops, and the solution has to be heated to −60° C. It is stirred for another hour at −70° C., and then the cold reaction solution is poured into a mixture of 2 kg of ice and 400 ml of concentrated hydrochloric acid. It is stirred vigorously and, after one hour, it is extracted several times with diethyl ether. The organic phase is washed neutral with water and dried on sodium sulfate. After concentration by evaporation, 49.3. g (188 mmol) of crude 4,5,6-trifluoro-2-N-pivaloylaminobenzaldehyde, which is combined with 26 g (275 mmol) of acetamidine hydrochloride, 38.3 g (277 mmol) of potassium carbonate and 30 g of molecular sieve (4A) in 206 ml of butyronitrile, is obtained. While being stirred vigorously, it is heated for 18 hours to 145° C., and the solvent is removed in a vacuum. After the residue is chromatographed on silica gel with hexane/ethyl acetate (0-100%), 9.1 g of 7,8-difluoro-5-N-pivaloylamino-2-methylquinazoline is obtained.

2.0 g (7.2 mmol) of 7,8-difluoro-5-N-pivaloylamino-2-methyquinazoline is dissolved in 140 ml of toluene and cooled to −70° C. Over 30 minutes, 24 ml (28.8 mmol) of a 1.2 M diisobutyl aluminum hydride solution in toluene is added in drops. The reaction mixture is allowed to heat over 2 hours to −25° C. and stirred for 2 hours at −25° C. Isopropanol and then water are slowly added and stirred for 12 hours at room temperature until a precipitate forms that is removed by means of filtration through Celite. It is rewashed well with a methylene chloride-methanol mixture and concentrated by evaporation. The residue is stirred vigorously in 200 ml of ethyl acetate and 50 ml of methanol together with 100 g of silica gel and 20 g of manganese dioxide. It is filtered through Celite, rewashed well with a methylene chloride-methanol mixture and concentrated by evaporation. After chromatography on silica gel (hexane-ethyl acetate 0-100%), 370 mg of the product is obtained.

¹H-NMR (300 MHz, CD₃OD); δ=2.81 (s, 3H), 6.64 (dd, 1H), 9.52 (s, 1H). p 158 mg (0.51 mmol) of 4-(3-fluoro-2-methoxyphenyl)-2-hydroxy-3-methyl-2-(trifluoromethyl)pentanal, 100 mg (0.51 mmol) of 5-amino-7,8-difluoro-2-methylquinazoline and 0.4 ml of titanium tetraethylate are stirred in 6 ml of toluene for 2 hours at 100° C. After cooling, it is poured into water, and vigorous stirring is continued. The suspension is filtered through Celite and thoroughly rewashed with ethyl acetate. The phases of the filtrate are separated, and it is extracted again with ethyl acetate. It is dried on sodium sulfate, the solvent is removed in a vacuum, and 1-[(7,8-difluoro-2-methylquinazolin-5-yl)-imino]-4-(3-fluoro-2-methoxyphenyl)-3methyl-2-(trifluoromethyl)-pentan-2-ol is obtained as a crude product. 5 ml (5 mmol) of a 1 M boron tribromide solution is added in drops at −35° C. over 10 minutes to imine in 10 ml of CH₂Cl₂. It is allowed to heat to −20° C. over 2 hours, and the batch is poured into cold saturated NaHCO₃ solution. It is rinsed with ethyl acetate, the phases are separated, the aqueous phase is extracted with ethyl acetate, the combined organic phases are dried (Na₂SO₄) and concentrated by evaporation in a vacuum. Column chromatography on silica gel (hexane/ethyl acetate/methanol 43:43:12%) and subsequent preparative thin-layer chromatography in an amino phase (Merck NH₂) provide 10 mg (0.02 mmol) of product as a racemic mixture of the main diastereomer.

¹H-NMR (300 MHz, CD₃OD): δ=1.17 (d, 3H), 1.45 (d, 3H), 2.44 (dq, 1H), 2.77 (s, 3H), 3.53 (dq, 1H), 5.10 (s, 1H), 6.69 (dd, 1H), 6.83 (dd, 1H), 6.86 (dd, 1H), 9.51 (s, 1H).

### EXAMPLE 7

### 2-Fluoro-5-[(2-methylquinolin-5-yl)amino]-5,6,7,8-tetrahydro-7,8-dimethyl-6-(trifluoromethyl)naphthalene-1,6-diol

The desired product can be produced analogously to Example 1 from 5-amino-2-methylquinoline and 4-(3-fluoro-2-methoxyphenyl)-2-hydroxy-3-methyl-2-(trifluoromethyl)pentanal.

### EXAMPLE 8

### 6-Chloro-5-methoxy-1-[(2-methylquinazolin-5-yl)amino]-3-methyl-4-methylene-2-(trifluoromethyl)-1,2,3,4-tetrahydronaphthalen-2-ol

### 4-(3-Chloro-2-methoxyphenyl)-2-hydroxy-3-methyl-2-(trifluoromethyl)pent-4-enal

265 mmol of propionic acid chloride is slowly added in drops to 250 mmol of 2-chlorophenol, dissolved in 200 ml of dichloromethane (DCM) and 38 ml of pyridine. After 2 hours of stirring, the batch is quenched with 1 M hydrochloric acid and extracted with DCM. The organic phases are washed with NaCl solution and dried with sodium sulfate. After the solvent is removed, 46 g of crude product, which is taken up in 40 ml of 1,2-dichlorobenzene, is obtained. This solution is added in drops at room temperature to a solution of 33 AlCl₃ in 40 ml of 1,2-dichlorobenzene. The mixture is stirred for 18 hours at 100° C., cooled, diluted with DCM, and added to ice/hydrochloric acid (4 m). The phases are separated, the aqueous phase is extracted with DCM, and the combined organic phases are washed with NaCl solution and dried with sodium sulfate. After the solvent is drawn off, 49 g of a mixture of the regioisomeric products is obtained. In the case of absorptive precipitation with hexane, 18 g of the para compound remains undissolved. The hexane solution is concentrated by evaporation. 22 g of this isomer-concentrated intermediate product is dissolved in acetone, 240 mmol of potassium carbonate and 144 mmol of methyl iodide are added carefully at room temperature, and it is stirred for 18 hours at 80° C. After cooling, the reaction mixture is added in water and extracted with diethyl ether. After drying with sodium sulfate and after the solvent is drawn off, the crude product is purified by column chromatography on silica gel (eluant:hexane/ethyl acetate 4:1). 18 g of the desired 1-(3-chloro-2-methoxy-phenyl)-propan-1-one is obtained.

56.2 g of zinc powder and 1.22 g of lead(II) chloride are introduced into 800 ml of THF, and 53 ml of dibromomethane is slowly added in drops at room temperature. After 30 minutes of stirring, 100 mmol of titanium(IV) chloride is slowly added in drops. After another 30 minutes of stirring, 100 mmol of 1-(3-chloro-2-methoxy-phenyl)-propan-1-one is added in drops and stirred for 4 hours at room temperature. It is diluted with diethyl ether, the reaction mixture is added to ice/hydrochloric acid (4 m), the phases are separated, extracted with diethyl ether and dried with sodium sulfate. The crude product solution is slowly concentrated in a rotary evaporator at a bath temperature of 45° C. and finally completely concentrated by evaporation. The crude product is chromatographed on silica gel with hexane/ethyl acetate 7:3. 16.7 g of 1-chloro-2-methoxy-3-(1-methylpropenyl)-benzene is obtained.

1.0 g of this styrene derivative is introduced with 1.30 g of trifluoropyruvate (1.5 equivalents) into 2.5 ml of dichloroethane, 82 mg (0.1 equivalent) of iron(III) chloride is added, and the mixture is heated for about 10 hours to 90° C. It is diluted with water and DCM, and the phases are separated. The aqueous phase is extracted with DCM, the combined organic phases are washed intensively with water and saturated NaCl solution and dried with sodium sulfate. After the solvent is separated, the crude product is purified by chromatography. 4-(3-Chloro-2-methoxyphenyl)-2-hydroxy-3-methyl-2-trifluoromethyl-4-pentenoic acid ethyl ester is obtained as a mixture of the isomers. 225 mg of this ester is mixed at −20° C. in 7 ml of THF with 24 mg of LiAlH₄. It is allowed to stir for about 4 hours at 0° C., another 20 mg of LiAlH₄ is added, and it is stirred for another 2 days. The mixture is poured into ice water, diluted with THF, ethyl acetate and water, and the phases are separated. The aqueous phase is made acidic with 2 M hydrochloric acid, extracted again, and the combined organic phases are washed with water and saturated NaCl solution and dried with sodium sulfate. After chromatographic purification, 209 mg of 4-(3-chloro-2-methoxy-phenyl)-3-methyl-2-trifluoromethyl-pent-4-ene-1,2-diol and 85 mg of 4-(3-chloro-2-methoxyphenyl)-2-hydroxy-3-methyl-2-trifluoromethyl-pent-4-enal are obtained. The diol that is obtained is dissolved in DCM, 0.22 ml of DMSO, 0.87 ml of triethylamine and 600 mg of sulfur trioxide-pyridine complex are added, and it is stirred for 7 hours at room temperature. The reaction mixture is dispersed between ammonium chloride solution and ethyl acetate, the phases are separated, extracted with ethyl acetate, washed with NaCl solution and dried with sodium sulfate. After chromatographic purification, 110 mg of additional 4-(3-chloro-2-methoxyphenyl)-2-hydroxy-3-methyl-2-trifluoromethyl-pent-4-enal is obtained as slightly yellowish oil.

113 mg of aldehyde is introduced into toluene with 83 mg (1.5 equivalents) of 5-amino-2-methylquinazoline, 0.18 ml of titanium tetraethylate is added in drops and refluxed for about 5 hours. After cooling, it is diluted with ethyl acetate, sodium bicarbonate solution is added, formed precipitates are filtered off over diatomaceous earth, and the phases are separated. The aqueous phase is extracted with ethyl acetate, and the combined organic phases are washed with saturated NaCl solution and dried with sodium sulfate. After the solvent is separated, the crude product is purified by chromatography. 60 mg of 4-(3-chloro-2-methoxy-phenyl)-3-methyl-1-[(2-methylquinazolin-5-yl)imino]-2-(trifluoromethyl)pent-4-en-2-ol is obtained as a mixture of the isomers. The imine is taken up in DCM, and 1.3 ml of titanium tetrachloride solution (1 M in DCM) is added in drops at −20° C. It is allowed to come to 0° C., and the mixture is added to ice water after 4 hours of stirring. It is extracted with DCM, washed intensively with water and dried with sodium sulfate. The crude product is filtered with DCM/MeOH over a little silica gel. 17 mg of 6-chloro-5-methoxy-3-methyl-4-methylene-1-(2-methylquinazolin-5-ylamino)-2-trifluoro-methyl-1,2,3,4-tetrahydronaphthalen-2-ol is obtained as an isomer mixture.

¹H-NMR (300 MHz, CDCl₃): δ (main isomer)=1.13 (d,3H), 2.88 (s,3H), 3.79 (s,3H), 5.02 (d,1H), 5.14 (d,1H), 5.80 (s,1H), 5.95 (s,1H), 6.89 (d,1H), 7.07 (m,1H), 7.19 (dd,1H), 7.38 (m,1H), 7.75 (m,1H), 9.45 (s,1H); MS (ESI): 464/466 (M+H), 482/484 (M+H+H₂O), 496/498 (M+H+MeOH).

In a Similar Way, There can be Produced:

a) 6-Chloro-1-[(7,8-difluoro-2-methylquinazolin-5-yl)amino]-5-methoxy-3-methyl-4-methylene-2-(trifluoromethyl)-1,2,3,4-tetrahydronaphthalen-2-ol

The desired product can be produced analogously to Example 8 from 5-amino-7,8-difluoro-2-methylquinazoline and 4-(3-chloro-2-methoxyphenyl)-2-hydroxy-3-methyl-2-trifluoromethyl-pent-4-enal.
b) 6-Chloro-5-methoxy-1-[(2-methylquinolin-5-yl)amino]-3-methyl-4-methylene-2-(trifluoromethyl)-1,2,3,4-tetrahydronaphthalen-2-ol

The desired product can be produced analogously to Example 8 from 5-amino-2-methylquinoline and 4-(3-chloro-2-methoxyphenyl)-2-hydroxy-3-methyl-2-trifluoromethyl-pent-4-enal.

### EXAMPLE 9

### 6,7,8,8a,9,10-Hexahydro-10-[(2-methyl-quinazolin-5-yl)amino]-9-(trifluoromethyl)-phenanthren-9-ol

### 3,3,3-Trifluoro-2-hydroxy-2-(2-phenyl-cyclohex-2-enyl)-propionic acid ethyl ester

870 mg (5.5 mmol) of 1-phenyl-1-cyclohexene, 1.87 g (11 mmol) and 310 mg (0.5 mmol) of ytterbium triflate are dissolved in 2 ml of 1,2-dichloroethane and refluxed for 19 hours. After cooling to room temperature, the solution is concentrated by evaporation in a vacuum and purified by column chromatography. 2 diastereomers are obtained: 312 mg of diastereomer A and 293 mg of diastereomer B.

¹H-NMR (300 MHz, CDCl₃): δ=1.07 (t, 3H), 1.59-1.67 (m, 1H), 1.72-1.85 (m, 1H), 2.10-2.34 (m, 4H), 3.01-3.11 (m, 1H), 3.57 (bs, 1H), 3.61-3.70 (m, 1H), 3.80 (s, 1H), 5.97 (t, 1H), 7.17-7.29 (m, 5H).

### 3,3,3-Trifluoro-2-hydroxy-2-(2-phenyl-cyclohex-2-enyl)-propionaldehyde

300 mg (0.91 mmol) of 3,3,3-trifluoro-2-hydroxy-2-(2-phenyl-cyclohex-2-enyl)-propionic acid ethyl ester is dissolved in 11 ml of diethyl ether and cooled under nitrogen atmosphere to −10° C. 69 mg (1.82 mmol) of lithium aluminum hydride is added in several portions. The reaction is stirred for 1 hour at −10° C. and for 1 hour at 0° C. For working-up, the reaction mixture is poured into saturated ammonium chloride solution, and the aqueous phase is extracted three times with ethyl acetate. The collected organic phases are washed with saturated sodium chloride solution, dried on sodium sulfate, filtered off, and concentrated by evaporation in a vacuum. The crude product that is obtained is purified by column chromatography, and 120 mg of product is obtained.

¹H-NMR (300 MHz, CDCl₃): δ=1.62-1.73 (m, 1H), 1.80-1.92 (m, 1H), 2.00-2.14 (m, 1H), 2.20-2.28 (m, 2H), 2.31-2.41 (m, 1H), 3.66 (bs, 1H), 3.78 (d, 1H), 6.05 (t, 1H), 7.14-7.20 (m, 2H), 7.24-7.33 (m, 3H), 9.05 (quint, 1H).

112 mg (0.39 mmol) of 3,3,3-trifluoro-2-hydroxy-2-(2-phenyl-cyclohex-2-enyl)-propionaldehyde, 62 mg (0.39 mmol) of 5-amino-2-methyl-quinazoline and 210 μl (1 mmol) of titanium tetraethylate were dissolved in 2 ml of toluene and refluxed under nitrogen atmosphere for 3 hours. After cooling to room temperature, the reaction mixture was poured into water, diluted with ethyl acetate, and filtered off over Celite. The aqueous phase is extracted twice with ethyl acetate. The collected organic phases are washed with saturated sodium chloride solution, dried on sodium sulfate, filtered and concentrated by evaporation in a vacuum. After purification by column chromatography on silica gel, 50 mg of 1,1,1-trifluoro-3-(2-methyl-quinazolin-5-yl-imino)-2-(-2-phenyl-cyclohex-2-enyl)-propan-2-ol is obtained.

¹H-NMR (400 MHz, CDCl₃): δ=1.70-1.78 (m, 1H), 1.81-1.91 (m, 1H), 2.26-2.39 (m, 3H), 2.54 (d, 1H), 2.91 (s, 3H), 3.67 (bs, 1H), 4.97 (s, 1H), 5.78 (d, 1H), 6.12 (t, 1H), 6.99-7.02 (m, 1H), 7.09 (t, 2H), 7.18-7.20 (m, 2H), 7.53 (t, 1H), 7.68 (s, 1H), 7.74 (d, 1H), 9.49 (s, 1H).

47 mg (0.11 mmol) of 1,1,1-trifluoro-3-(2-methyl-quinazolin-5-yl-imino)-2-(2-phenyl-cyclohex-2-enyl)-propan-2-ol is dissolved in 2 ml of dichloromethane and cooled under nitrogen atmosphere to −20° C. 440 μl (0.44 mmol, 1 M solution in dichloromethane) is slowly added to the reaction. The mixture is stirred for 2 hours at room temperature. For working-up, it is poured into saturated sodium bicarbonate solution and extracted three times with ethyl acetate. The collected organic phases are washed with saturated sodium chloride solution, dried on sodium sulfate, filtered, and concentrated by evaporation in a vacuum. The crude product is purified by column chromatography and with preparative HPLC. 20 mg of product is obtained.

¹H-NMR (400 MHz, DMSO-d₆): δ=1.40 (bs, 1H), 1.80-18.7 (m, 2H), 1.94 (bs, 1H), 2.20 (bs, 2H), 2.69 (s, 3H), 3.04 (bs, 1H), 5.62 (d, 1H), 6.11 (s, 1H), 6.76 (bs, 1H), 6.90 (d, 1H), 7.00 (d, 1H), 7.05 (d, 1H), 7.17 (t, 1H), 7.23-7.25 (m, 2H), 7.71-7.25 (m, 2H), 9.63 (s, 1H).

In a Similar Way, There can be Produced:

a) 10-[(7,8-Difluoro-2-methyl-quinazolin-5-yl)amino]-6,7,8,8a,9,10-hexahydro-9-(trifluoromethyl)-phenanthren-9-ol

The desired product can be produced analogously to Example 9 from 5-amino-7,8-difluoro-2-methylquinazoline and 3,3,3-trifluoro-2-hydroxy-2-(2-phenyl-cyclohex-2-enyl)-propionaldehyde.
b) 5-{[10-[6,7,8,8a,9,10-Hexahydro-9-(trifluoromethyl)-9-hydroxy-phenanthrenyl]amino}quinolin-2(1H)-one

The desired product can be produced analogously to Example 9 from 5-aminoquinolin-2(1H)-one and 3,3,3-trifluoro-2-hydroxy-2-(2-phenyl-cyclohex-2-enyl)-propionaldehyde.
c) 6,7,8,8a,9,10-Hexahydro-10-[(2-methyl-quinolin-5-yl)amino]-9-(trifluoromethyl)-phenanthren-9-ol

The desired product can be produced analogously to Example 9 from 5-amino-2-methyl-quinoline and 3,3,3-trifluoro-2-hydroxy-2-(2-phenyl-cyclohex-2-enyl)-propionaldehyde.
d) 3,3a,4,5-Tetrahydro-5-[(2-methyl-quinazolin-5-yl)amino]-4-(trifluoromethyl)-2H-benz[e]inden-4-ol

The desired product can be produced analogously to Example 9 from 5-amino-2-methyl-quinoline and 1-phenyl-1-cyclopentene.
e) 5-[(7,8-Difluoro-2-methyl-quinazolin-5-yl)amino]-3,3a,4,5-tetrahydro-4-(trifluoromethyl)-2H-benz[e]inden-4-ol

The desired product can be produced analogously to Example 9 from 5-amino-7,8-difluoro-2-methyl-quinazoline and 1-phenyl-1-cyclopentene.

### EXAMPLE 10

### 4b,5,6,7,8,8a,9,10-Octahydro-10-[(2-methylquinazolin-5-yl)amino]-9-(trifluoromethyl)-phenanthren-9-ol

### 3,3,3-Trifluoro-2-hydroxy-2-(2-phenyl-cyclohexyl)-propionaldehyde

500 mg (1.52 mmol) of 3,3,3-trifluoro-2-hydroxy-(2-phenyl-cyclohex-2-enyl)-propionic acid ethyl ester is dissolved in 20 ml of methanol and provided with 300 μl of acetic acid and 30 mg of palladium on activated carbon (10%). The flask is evacuated and then filled with hydrogen. The reaction was stirred for 5 hours at room temperature. For working-up, it is filtered over Celite and concentrated by evaporation in a vacuum. 492 mg of crude 3,3,3-trifluoro-2-hydroxy-2-(2-phenyl-cyclohexyl-propionic acid ethyl ester, which is incorporated directly into the subsequent reaction without further purification, is obtained.

¹H-NMR (400 MHz, CDCl₃): δ=1.23 (t, 3H), 1.41-1.59 (m, 2H), 1.70-1.87 (m, 3H), 1.90-2.00 (m, 1H), 2.09-2.19 (m, 1H), 2.20-2.29 (m, 1H), 2.73-2.77 (m, 1H), 2.91-2.95 (m, 1H), 3.32 (s, 1H), 3.78-3.87 (m, 1H), 4.07-4.09 (m, 1H), 7.17-7.28 (m, 5H).

485 mg (1.47 mmol) of 3,3,3-trifluoro-2-hydroxy-2-(2-phenylcyclohexyl)-propionic acid ethyl ester is dissolved in 18 ml of diethyl ether and cooled under nitrogen atmosphere to −10° C. 111 mg (2.94 mmol) of lithium aluminum hydride is added in several portions, stirred for 1 hour at −10° C., and it is allowed to thaw over 3 hours to room temperature. For working-up, it is poured into saturated ammonium chloride solution and extracted three times with ethyl acetate. The combined organic phases are washed with saturated sodium chloride solution, dried on sodium sulfate, filtered off and concentrated by evaporation in a vacuum. The crude product was purified by column chromatography on silica gel. 200 mg of product is obtained.

¹H-NMR (400 MHz, CDCl₃): δ=1.40-2.20 (m, 8H), 2.82-2.86 (m, 1H), 3.05-3.09 (m, 1H), 3.58 (s, 1H), 7.14-7.31 (m, 5H), 8.87 (s, 1H).

190 mg (0.66 mmol) of 3,3,3-trifluoro-2-hydroxy-2-(2-phenyl-cyclohexyl)-propionaldehyde, 106 mg (0.66 mmol) of 5-amino-2-methyl-quinazoline and 350 μl (1.7 mmol) of titanium tetraethylate are dissolved in 3.5 ml of toluene and refluxed for 3 hours under nitrogen atmosphere. After cooling to room temperature, the reaction mixture is poured into water, diluted with ethyl acetate and filtered off over Celite. The aqueous phase is extracted twice with ethyl acetate. The collected organic phases are washed with saturated sodium chloride solution, dried on sodium sulfate, filtered and concentrated by evaporation in a vacuum. After purification by column chromatography, 69 mg of 1,1,1-trifluoro-3-(2-methyl-quinazolin-5-ylimino)-(2-phenyl-cyclohexyl)-propan-2-ol is obtained.

¹H-NMR (400 MHz, CDCl₃): δ=1.44-2.16 (m, 8H), 2.74-2.81 (m, 1H), 2.92 (s, 3H), 3.29 (q, 1H), 4.77 (s, 1H), 6.61 (dd, 1H), 7.04-7.12 (m, 3H), 7.33 (d, 2H), 7.53 (s, 1H), 7.70 (t, 1H), 7.83 (d, 1H), 9.37 (s, 1H).

66 mg (0.15 mmol) of 1,1,1-trifluoro-3-(2-methyl-quinazolin-5-yl-imino)-2-(-2-phenyl-cyclohex-2-enyl)-propanol is dissolved in 3 ml of dichloromethane and cooled under nitrogen atmosphere to −20° C. 620 μl of a boron tribromide solution (0.62 mmol, 1 M solution in dichloromethane) is slowly added to the reaction. The mixture is stirred for 2 hours at room temperature. For working-up, it is poured into saturated sodium bicarbonate solution and extracted three times with ethyl acetate. The collected organic phases are washed with saturated sodium chloride solution, dried on sodium sulfate, filtered, and concentrated by evaporation in a vacuum. The crude product is purified by column chromatography, and 33 mg of the desired product is obtained.

¹H-NMR (500 MHz, CDCl₃): δ=0.83-0.93 (m, 1H), 1.41-1.75 (m, 6H), 1.83-1.95 (m, 2H), 2.57-2.65 (m, 2H), 2.85 (s, 3H), 3.18 (bs, 1H), 4.78 (d, 1H), 5.78 (d, 1H), 6.46 (d, 1H), 7.21 (t, 1H), 7.29 (t, 1H), 7.37 (t, 1H), 7.45 (d, 1H), 7.61 (t, 1H), 9.43 (s, 1H).

In a Similar Way, There can be Produced:

a) 10-[(7,8-Difluoro-2-methyl-quinazolin-5-yl)amino]-4b,5,6,7,8,8a,9,10-octahydro-9-(trifluoromethyl)-phenanthren-9-ol

The desired product can be produced analogously to Example 10 from 5-amino-7,8-difluoro-2-methylquinazoline and 3,3,3-trifluoro-2-hydroxy-2-(2-phenyl-cyclohexyl)-propionaldehyde.
b) 5-{[10-[4b,5,6,7,8,8a,9,10-Octahydro-9-hydroxy-9-(trifluoromethyl)-phenanthrenyl]amino}-quinolin-2(1H)-one

The desired product can be produced analogously to Example 10 from 5-aminoquinolin-2(1H)-one and 3,3,3-trifluoro-2-hydroxy-2-(2-phenyl-cyclohexyl)-propionaldehyde.
c) 4b,5,6,7,8,8a,9,10-Octahydro-10-[(2-methyl-quinolin-5-yl)amino]-9-(trifluoromethyl)-phenanthren-9-ol

The desired product can be produced analogously to Example 10 from 5-amino-2-methyl-quinoline and 3,3,3-trifluoro-2-hydroxy-2-(2-phenyl-cyclohexyl)-propionaldehyde.
d) 2,3,3a,4,5 9b-Hexahydro-5-[(2-methyl-quinazolin-5-yl)amino]-4-(trifluoromethyl)-1H-benz[e]inden-4-ol

The desired product can be produced analogously to Example 10 from 5-amino-2-methyl-quinazoline and 1-phenyl-1-cyclopentene.
e) 5-[(7,8-Difluoro-2-methyl-quinazolin-5-yl)amino]-2,3,3a,4,5,9b-hexahydro-4-(trifluoromethyl)-1H-benz[e]inden-4-ol

The desired product can be produced analogously to Example 10 from 5-amino-7,8-difluoro-2-methyl-quinazoline and 1-phenyl-1-cyclopentene.

### EXAMPLE 11

### (3aα,4α,5α9bα) 2,3,3a,4,5,9b-Hexahydro-8-fluoro-5-[(2-methylquinazolin-5-yl)amino]-4-(trifluoromethyl)-1H-benz[e]indene-4,9-diol

### (1α,2α)-2-(3-Fluoro-2-methoxyphenyl)-α-hydroxy-α-(trifluoromethyl)cyclopentane acetaldehyde

2.62 g (15.5 mmol) of 3-fluoro-2-methoxyphenyl)boronic acid, 7.14 g of (nonafluorobutyl)sulfonyl acid cyclopent-1-enyl ester, 720 mg (42 mmol) of lithium chloride, 2.68 g (19.4 mmol) of potassium carbonate and tetrakis(triphenylphosphine)-palladium are dissolved in 8 ml of toluene and 8 ml of 1-propanol. The mixture is heated over 6 hours to 110° C. and added after cooling in water. The aqueous phase is extracted three times with ethyl acetate, the combined organic phases are washed with saturated sodium chloride solution and dried on Na₂SO₄. After the solvent is removed, the residue is purified by column chromatography on silica gel (hexane/diisopropyl ether, 0-10%). 1.9 g of 6-(cyclopent-1-enyl)-2-fluoroanisole is obtained. 2.6 ml (19.7 mmol) of ethyltrifluoropyruvate and 1.9 g (9.9 mmol) of 6-(cyclopent-1-enyl)-2-fluoroanisole in 10 ml of dichloromethane are added to 613 mg (0.99 mmol) of ytterbium trifluoromethane sulfonate. The reaction mixture is stirred for 16 hours at 140° C., and the reaction mixture is purified by means of column chromatography on silica gel (hexane/diisopropyl ether 0-10%). 391 mg of ethyl-2-(3-fluoro-2-methoxyphenyl)-α-hydroxy-α-(trifluoromethyl)cyclopent-2-ene acetate is obtained as a mixture of diastereomers. 211 mg (0.58 mmol) of ethyl-2-(3-fluoro-2-methoxy-phenyl)-α-hydroxy-α-(trifluoromethyl)cyclopent-2-ene acetate is dissolved in 18 ml of diethyl ether under argon and cooled to −25° C. 52 mg (1.4 mmol) of lithium aluminum hydrides is added in portions as a solid, and it is stirred for 1.5 hours, whereby the temperature increases to −20° C. 1 ml of ethyl acetate is added, poured after 10 minutes into a mixture of ice and saturated ammonium chloride solution and stirred vigorously. The phases are separated and extracted several times with ethyl acetate and diethyl ether. The combined organic extracts are filtered through Celite, washed with saturated sodium chloride solution and dried on Na₂SO₄. 182 mg of 2-(3-fluoro-2-methoxyphenyl)-α-hydroxy-α-(trifluoromethyl)cyclopent-2-ene acetaldehyde is obtained.

114 mg (0.36 mmol) of 32-(3-fluoro-2-methoxyphenyl)-α-hydroxy-α-(trifluoromethyl)cyclopent-2-ene acetaldehyde is dissolved in 11 ml of methanol and 0.1 ml of acetic acid, and 20 mg of palladium on carbon (10%) is added. The suspension is shaken under a hydrogen atmosphere at normal pressure until the reaction is completed. The mixture is filtered through Celite, and it is rewashed thoroughly with ethyl acetate. After the removal of the solvent, 108 mg of the saturated aldehyde is obtained as a mixture of 2 diastereomers. The diastereomers can be separated from one another by column chromatography on silica gel (hexane/diisopropyl ether 0-15%): [1α(S*),2α]-2-(3-fluoro-2-methoxyphenyl)-α-hydroxy-α-(trifluoromethyl)-cyclopentane acetaldehyde:

¹H-NMR (300 MHz, CDCl₃); δ=1.78 (m, 2H), 1.98 (m, 2H), 2.15 (m, 3H), 2.88 (ddd, 1H), 3.95 (s, 3H), 3.97 (s, 1H), 4.46 (s, 1H), 6.84-6.99 (m, 3H), 9.06 (s, 1H).

[1α(R*),2α]-2-(3-Fluoro-2-methoxyphenyl)-α-hydroxy-α-(trifluoromethyl)-cyclopentane acetaldehyde: ¹H-NMR (300 MHz, CDCl₃); δ=1.60 (m, 2H), 1.80-2.35 (m, 5H), 3.26 (ddd, 1H), 3.62 (s, 1H), 3.95 (s, 3H), 6.84-6.99 (m, 3H), 9.18 (s, 1H).

117 mg (0.37 mmol) of [1α(S*),2α]-2-(3-fluoro-2-methoxyphenyl)-α-hydroxy-α-(trifluoromethyl)cyclopentane acetaldehyde, 65 mg (0.41 mmol) of 5-amino-2-methylquinazoline and 0.2 ml of titanium tetraethylate are stirred in 10 ml of toluene for 2 hours at 100° C. After cooling, it is poured into water, and vigorous stirring is continued. The suspension is filtered through Celite and thoroughly rewashed with ethyl acetate. The phases of the filtrate are separated, and it is extracted again with ethyl acetate. It is dried on sodium sulfate, and the solvent is removed in a vacuum, and 116 mg of [1α(S*),2α]-2-(3-fluoro-2-methoxyphenyl)-α-{[(methylquinazolin-5yl)imino]methyl}-α-(trifluoromethyl)-cyclopentane methanol is obtained as a crude product. 2.2 ml (2.2 mmol) of a 1 M boron tribromide solution is added in drops to 116 mg of crude imine in 20 ml of CH₂Cl₂ at −30° C. It is allowed to heat to room temperature and stirred for 12 hours. It is poured into saturated NaHCO₃ solution, the phases are separated, the aqueous phase is extracted with CH₂Cl₂, the combined organic phases (Na₂SO₄) are dried and concentrated by evaporation in a vacuum. Column chromatography on silica gel (hexane/ethyl acetate 0-80%) yields 62 mg of product.

¹H-NMR (300 MHz, DMSO [d6]); δ=1.32 (m, 1H), 1.46 (m, 1H), 1.67 (m, 2H), 1.76 (m, 1H), 2.42 (m, 1H), 2.50 (m, 1H), 2.67 (s, 3H), 3.28 (d, 1H), 3.57 (ddd, 1H), 5.14 (d, 1H), 6.01 (s, 1H), 6.61 (dd, 1H), 6.89 (m, 2H), 7.05 (d, 1H), 7.63 (t, 1H), 9.41 (s, 1H), 9.65 (s, 1H).

### EXAMPLE 12

### (3aα,4β,5β,9b α)-2,3,3a,4,5,9b-Hexahydro-8-fluoro-5-[(2-methylquinazolin-5-yl)amino]-4-(trifluoromethyl)-1H-benz[e]indene-4,9-diol

65 mg (0.2 mmol) of [1α(R*),2α]-2-(3-fluoro-2-methoxyphenyl)-α-hydroxy-α-(trifluoromethyl)cyclopentane acetaldehyde, 36 mg (0.23 mmol) of 5-amino-2-methylquinazoline and 0.11 ml of titanium tetraethylate are stirred in 5 ml of toluene for 2 hours at 100° C. After cooling, it is poured into water, and vigorous stirring is continued. The suspension is filtered through Celite and rewashed thoroughly with ethyl acetate. The phases of the filtrate are separated, and it is extracted again with ethyl acetate. It is dried on sodium sulfate and the solvent is removed in a vacuum, and 100 mg of [1α(R*),2α]-2-(3-fluoro-2-methoxyphenyl)-α-{[(methylquinazolin-5yl)imino]methyl}-α-(trifluoromethyl)-cyclopentane methanol is obtained as a crude product. 1.9 ml (1.9 mmol) of a 1 M boron tribromide solution is added in drops to 100 mg of crude imine in 12 ml of CH₂Cl₂ at −30° C. It is allowed to heat to room temperature and stirred for 2 hours. It is poured into saturated NaHCO₃ solution, the phases are separated, the aqueous phase is extracted with CH₂Cl₂, the combined organic phases (Na₂SO₄) are dried and concentrated by evaporation in a vacuum. Column chromatography on silica gel (hexane/ethyl acetate 0-75%) yields 43 mg of product.

¹H-NMR (300 MHz, DMSO [d6]); δ=1.38 (m, 2H), 1.74 (m, 1H), 1.85 (m, 1H), 2.11 (m, 1H), 2.64 (m, 1H), 2.67 (s, 3H), 2.78 (ddd, 1H), 3.12 (d, 1H), 3.16 (ddd, 1H), 5.29 (d, 1H), 5.99 (s, 1H), 6.61 (dd, 1H), 6.72 (d, 1H), 6.90 (m, 2H), 7.01 (d, 1H), 7.60 (t, 1H), 9.46 (s, 1H), 9.67 (s, 1H).

### EXAMPLE 13

### (3aα,4α,5α,9bα)-8-Fluoro-5-[(7-fluoro-2-methylquinazolin-5-yl)amino]-2,3,3a,4,5,9b-hexahydro-4-(trifluoromethyl)-1H-benz[e]indene-4,9-diol

Analogously to Example 11, 108 mg (0.34 mmol) of [1α(S*),2α]-2-(3-fluoro-2-methoxyphenyl)-α-hydroxy-α-(trifluoromethyl)cyclopentane acetaldehyde, 66 mg (0.37 mmol) of 5-amino-7-fluoro-2-methylquinazoline and 0.2 ml of titanium tetraethylate are reacted to form the corresponding imine. 156 mg of thus obtained crude imine is cyclized analogously to Example 11 whereby addition is done at a temperature of −30° C. and then for 2 hours at room temperature with 2.8 ml (2.8 mmol) of 1 M boron tribromide solution to form the desired product. Chromatography on silica gel (hexane/ethyl acetate 0-80%) yields 66 mg of the desired product.

¹H-NMR (300 MHz, CD₃OD); δ=1.49 (m, 2H), 1.70 (m, 1H), 1.80 (m, 1H), 1.94 (m, 1H), 2.57 (m, 1H), 2.64 (ddd, 1H), 2.75 (s, 3H), 3.72 (ddd, 1H), 5.13 (s, 1H), 6.65 (dd, 1H), 6.66 (d, 1H), 6.76 (d, 1H), 6.84 (dd, 1H), 9.50 (s, 1H).

### EXAMPLE 14

### (3aα,4β,5β,9bα)-8-Fluoro-5-[(7-fluoro-2-methylquinazolin-5-yl)amino]-2,3,3a,4,5,9b-hexahydro-4-(trifluoromethyl)-1H-benz[e]indene-4,9-indole

Analogously to Example 12, 130 mg (0.4 mmol) of [1α(R*),2α]-2-(3-fluoro-2-methoxyphenyl)-α-hydroxy-α-(trifluoromethyl)cyclopentane acetaldehyde, 79 mg (0.44 mmol) of 5-amino-7-fluoro-2-methylquinazoline and 0.22 ml of titanium tetraethylate are reacted to form the corresponding imine. 205 mg of the thus obtained crude imine is cyclized with 3.7 ml (3.7 mmol) of 1 M boron tribromide solution to the desired product analogously to Example 12 whereby addition is done at a temperature of −30° C. and then for 2 hours at room temperature. Chromatography on silica gel (hexane/ethyl acetate 0-75%) yields 49 mg of the desired product.

¹H-NMR(300 MHz, CD₃OD); δ=1.52 (m, 2H), 1.84 (m, 1H), 1.97 (m, 1H), 2.22 (m, 1H), 2.70-2.86 (m, 2H), 2.74 (s, 3H), 3.35 (ddd, 1H), 5.17 (s, 1H), 6.61 (d, 1H), 6.65 (dd, 1H), 6.74 (d, 1H), 6.84 (dd, 1H), 9.50 (s, 1H).

### EXAMPLE 15

### 5-{[(3aα,4α,5α,9bα)-8-Fluoro-2,3,3a,4,5,9b-hexahydro-4,9-dihydroxy-4-(trifluoromethyl)-1H-benz[e]inden-5-yl]amino}quinolin-2(1H)-one

Analogously to Example 11, 108 mg (0.34 mmol) of [1α(S*),2α]-2-(3-fluoro-2-methoxyphenyl)-α-hydroxy-α-(trifluoromethyl)cyclopentane acetaldehyde, 66 mg (0.37 mmol) of 5-aminoquinolin-2(1H)-one and 0.18 ml of titanium tetraethylate are reacted to form the corresponding imine. 147 mg of the thus obtained crude imine is cyclized to the desired product with 2.8 ml (2.8 mmol) of 1 M of boron tribromide solution, at a temperature of addition, analogously to Example 11, whereby addition is done at a temperature of −30° C. and then for 2 hours at room temperature. Chromatography on silica gel (hexane/ethyl acetate 0-100%) yields 72 mg of the desired product.

¹H-NMR (300 MHz, CD₃OD); δ=1.45 (m, 1H), 1.57 (m, 1H), 1.64 (m, 1H), 1.77 (m, 1H), 1.95 (m, 1H), 2.55 (m, 1H), 2.63 (ddd, 1H), 3.70 (ddd, 1H), 5.04 (s, 1H), 6.48 (d, 1H), 6.52 (d, 1H), 6.65 (d, 1H), 6.66 (dd, 1H), 6.80 (dd, 1H), 7.32 (t, 1H), 8.19 (d, 1H).

### EXAMPLE 16

### 5-{[6-Fluoro-2,5-dihydroxy-3,4-dimethyl-2-(trifluoromethyl)-1,2,3,4-tetrahydronaphthalen-1-yl]amino}isochromen-1-one

With the aid of the aldehyde that is described in Example 1, the imine is produced as usual. After cyclization with boron tribromide, 38.3 mg of the nonpolar diastereomer of 5-{[6-fluoro-2,5-dihydroxy-3,4-dimethyl-2-(trifluoromethyl)-1,2,3,4-tetrahydronaphthalen-1-yl]amino}isochromen-1-one and 9.1 mg of the polar diastereomer of 5-{[6-fluoro-2,5-dihydroxy-3,4-dimethyl-2-(trifluoromethyl)-1,2,3,4-tetrahydronaphthalen-1-yl]amino}isochromen-1-one are obtained.

Nonpolar diastereomer: ¹H-NMR (300 MHz, CD₃OD); δ=1.18 (3H), 1.45 (3H), 2.39 (1H), 3.51 (1H), 5.03 (1H), 6.63-6.72 (1H), 6.80 (1H), 6.87 (1H), 7.19 (1H), 7.32 (1H), 7.41 (1H), 7.58 (1H).

Polar diastereomer: ¹H-NMR (300 MHz, CD₃OD); δ=1.28 (3H), 1.43 (3H), 2.32 (1H), 3.20 (1H), 5.09 (1H), 6.63-6.72 (1H), 6.79 (1H), 6.85 (1H), 7.20 (1H), 7.35 (1H), 7.40 (1H), 7.55 (1H).

### EXAMPLE 17

### 8-Fluoro-5-{[6-fluoro-2,5-dihydroxy-3,4-dimethyl 6-(trifluoromethyl)-1,2,3,4-tetrahydronaphthalen-1-yl]amino}quinolin-2(1H)-one

### 5-Amino-8-fluoroquinolin-2(1H)-one

7.8 g (43 mmol) of 5,8-difluoroquinolin-2(1H)-one and 1.18 g (8.2 mmol) of Cu₂O are mixed with gaseous NH₃ in 620 ml of ethylene glycol under 8 bar. The reaction mixture is heated for 19 hours to 190° C. After the reaction mixture is cooled and after the solvent is removed, the crude product is purified by column chromatography (silica gel, hexane; CH₂Cl₂/MeOH 0-5%). 1.03 g of 5-amino-8-fluoroquinolin-2-(1H)-one is obtained as a light yellow solid.

¹H-NMR (300 MHz, DMSO-d6); δ=5.58 (s, 2H), 6.23 (dd, 1H), 6.31 (d, 1H), 7.03 (dd, 1H), 8.05 (dd, 1H), 11.28 (s, 1H).

The desired product can be produced analogously to Example 15 from 5-amino-8-fluoroquinolin-2(1H)-one and 4-(3-fluoro-2-methoxyphenyl)-2-hydroxy-3-methyl-2-(trifluoromethyl)pentanal.

¹H-NMR (400 MHz, CD₃OD); δ=1.18 (d, 3H), 1.48 (d, 3H), 2.45 (m, 1H), 3.55 (m, 1H), 5.04 (s, 1H), 6.53 (d, 1H), 6.58 (dd, 1H), 6.73 (m, 1H), 6.87 (dd, 1H), 7.24 (dd, 1H), 8.16 (d, 1H).

### EXAMPLE 18

### 5-{[6-Fluoro-2,5-dihydroxy-3,4-dimethyl 2-(trifluoromethyl)-1,2,3,4-tetrahydronaphthalen-1-yl]amino}isoquinolin-1(2H)-one

The desired product can be produced analogously to Example 15 from 5-aminoisoquinolin-1(2H)-one and 4-(3-fluoro-2-methoxyphenyl)-2-hydroxy-3-methyl-2-(trifluoromethyl)pentanal:

Nonpolar diastereomer: ¹H-NMR (300 MHz, CD₃OD), δ=1.29 (3H), 1.44 (3H), 2.35 (1H), 3.20 (1H), 5.09 (1H), 6.69-6.87 (3H), 7.10 (2H), 7.38 (1H), 7.66 (1H).

Polar diastereomer: ¹H-NMR (300 MHz, DMSO [d6]), polar δ=1.02 (3H), 1.35 (3H), 2.31 (1H), 3.43 (1H), 5.05 (1H), 5.78 (1H), 6.23 (1H), 6.51-6.61 (1H), 6.69 (1H), 6.91 (1H), 7.02-7.15 (2H), 7.26 (1H), 7.49 (1H), 9.4 (1H), 11.18 (1H).

### EXAMPLE 19

### 5-{[7-Fluoro-2,5-dihydroxy-3,4-dimethyl 2-(trifluoromethyl)-1,2,3,4-tetrahydronaphthalen-1-yl]amino}isoquinolin-1(2H)-one

### 1-(4-Fluoro-2-hydroxyphenyl)propan-1-one

100 g (892 mmol) of 3-fluorophenol is dissolved in 1.4 L of pyridine and mixed drop by drop at 0° C. with 99 g (1.07 mol) of propionyl chloride. After stirring overnight at room temperature, the mixture is poured into two liters of ice water and extracted three times with methyl-tert-butyl ether. The combined organic extracts are washed with water and with saturated brine, dried on sodium sulfate, and the solvent is spun off. The remaining residue is chromatographed on silica gel (mobile solvent ethyl acetate/hexane). 148.7 g (99.1%) of propionic acid-3-fluorophenyl ester is isolated.

¹H-NMR (400 MHz, CDCl₃): δ=1.29 (3H), 2.60 (2H), 6.81-6.69 (3H), 7.30-7.39 (1H).

15.38 g (115.36 mmol) of aluminum trichloride is introduced into 48 ml of 1,2-dichlorobenzene. At room temperature, 20 g (118.93 mmol) of propionic acid-3-fluorophenyl ester, dissolved in 41 ml of 1,2-dichlorobenzene, is now added in drops. In this case, a slight warming occurs. The reaction mixture is then stirred for 18 hours at 100° C. After cooling, the batch is added to 4N hydrochloric acid and extracted three times with methyl-tert-butyl ether. The combined organic extracts are washed with brine, dried, and the solvent is spun off. After the residue is chromatographed on silica gel (mobile solvent ethyl acetate/hexane), 16.75 g (83.75%) of the desired product is obtained.

¹H-NMR (300 MHz, CDCl₃): δ=1.25 (3H), 3.00 (2H), 6.55-6.70 (2H), 7.70-7.83 (1H), 12.7 (1H).

### Ethyl-4-(4-fluoro-2-methylphenyl)-2-hydroxy-3-methyl-2-(trifluoromethyl)-pent-4-enoate

16.75 g (99.6 mmol) of 1-(4-fluoro-2-hydroxyphenyl)propan-1-one is mixed in 124 ml of acetone with 27.53 g (199.2 mmol) of potassium carbonate and 28.27 g (199.2 mmol) of iodomethane. After four hours of reflux boiling, the mixture is filtered through a glass fiber filter, and the filtrate is spun in. The remaining residue is chromatographed on silica gel (mobile solvent ethyl acetate/hexane). 16.84 g (92.8%) of 1-(4-fluoro-2-methoxyphenyl)-propan-1-one is isolated:

¹H-NMR (300 MHz, CDCl₃); δ=1.18 (3H), 2.97 (2H), 3.91 (3H), 6.62-6.76 (2H), 7.79 (1H).

56 g (858.4 mmol) of Zn and 1.04 g (3.73 mmol) of PbCl₂ are introduced at room temperature into 545 ml of THF. 111.43 g (641 mmol) of dibromomethane is slowly added in drops to this mixture, and the batch is stirred for 30 minutes at room temperature. After cooling to 0° C., 93.3 ml of a one-molar solution of titanium(IV) chloride solution in dichloromethane is slowly added in drops, specifically so that the temperature does not increase beyond 10° C. After 30 minutes of stirring at room temperature, the batch is again cooled to 0° C., and 17 g (93.21 mmol) of the previously described 1-(4-fluoro-2-methoxyphenyl)-propan-1-one, dissolved in 75 ml of THF, is added in drops. After one hour of stirring at room temperature (heating to about 28° C., water bath cooling), the reaction mixture is diluted with 150 ml of diethyl ether and then carefully added to 4N HCl. In this case, the temperature is kept at 28-30° C. and stirred vigorously for 15 minutes. After three cycles of extraction with diethyl ether, the combined organic phases are washed twice with brine, dried on sodium sulfate, and the solvent is spun off. The crude product is chromatographed on the same date on silica gel (mobile solvent ethyl acetate/hexane), and the 4-fluoro-2-methoxy-1-(1-methylenepropyl)benzene that is obtained (9.58 g=56.97%) is stored in a refrigerator.

¹H-NMR (300 MHz, CDCl₃): δ=1.00 (3H), 2.45 (2H), 3.81 (3H), 4.98 (1H), 5.15 (1H), 6.52-6.78 (2H), 7.09 (1H).

3.3 g (5.32 mmol) of ytterbium(III) trifluoromethanesulfonate is introduced into 118 ml of dichloromethane. After 18.08 g (106.31 mmol) of ethyltrifluoropyruvate is added in drops, 9.58 g (53.16 mmol) of the previously described alkene, dissolved in 30 ml of dichloromethane, is added in drops, and the batch is then stirred for two days at room temperature. For working-up, the reaction mixture is mixed with 25 ml of water, and the organic phase is separated. After drying on sodium sulfate, the solvent is spun off, and the residue is chromatographed on silica gel (mobile solvent ethyl acetate/hexane). 14.33 g (76.96%) of a mixture that consists of (E/Z)-ethyl-4-(4-fluoro-2-methoxyphenyl)-2-hydroxy-2-(trifluoromethyl)-hex-4-enoate and ethyl-4-(4-fluoro-2-methoxyyphenyl)-2-hydroxy-3-methyl-2-(trifluoromethyl)-pent-4-enoate is obtained, which thus is incorporated into the next reaction.

### 4-(4-Fluoro-2-methoxyphenyl)-2-hydroxy-3-methyl-2-(trifluoromethyl)pentanal

14.3 g (40.9 mmol) of the mixture that is obtained from the previously described reaction step is dissolved in 320 ml of diethyl ether and mixed in portions with 1.55 g (40.91 mmol) of lithium aluminum hydride at 0 to 5° C. After four hours of stirring at room temperature, and after the batch is cooled to a temperature of between 0 and 5° C., 30 ml of saturated sodium bicarbonate solution is carefully added in drops. After 30 minutes of vigorous stirring, the batch is suctioned off through a glass fiber filter. The filtrate is extracted three times with methyl-tert-butyl ether, and the combined organic extracts are washed with brine, dried on sodium sulfate, and the solvent is spun off. After the residue is chromatographed on silica gel (mobile solvent ethyl acetate/hexane), 11.03 g (87.47%) of the desired compound is obtained as a mixture that consists of (E/Z) 4-(4-fluoro-2-methoxyphenyl)-2-(trifluoromethyl)-hex-4-ene-1,2-diol and 4-(4-fluoro-2-methoxyphenyl)-3-methyl-2-(trifluoromethyl)-pent-4-ene-1,2-diol. MS (Cl) m/e (relative intensity); 326 (100%)

4.86 g (15.76 mmol) of the mixture that is described in the preceding section is dissolved in a 1:1 mixture that consists of tetrahydrofuran and ethanol (altogether 486 ml) and mixed with 0.5 g (1.8 mmol) of Pd/calcium carbonate. After 335 ml of hydrogen is taken up and after a reaction time of 2¼ hours, the catalyst is filtered off and the filtrate is spun off. After the residue is chromatographed on a Flashmaster (mobile solvent ethyl acetate/hexane), 4.46 g (89.13%) of the hydrogenated compounds is obtained. 1 g (3.22 mmol) of the mixture that consists of 4-(4-fluoro-2-methoxyphenyl)-2-(trifluoromethyl)-hexane-1,2-diol and 4-(4-fluoro-2-methoxyphenyl)-3-methyl-2-(trifluoromethyl)-pentane-1,2-diol are oxidized under Swern conditions at −70° C. For working-up, 2.46 ml of triethylamine is added in drops thereto always at −70° C., and the batch is brought to room temperature. After 10 ml of water is added, it is stirred for 15 minutes. The dichloromethane phase is separated, and the aqueous phase is extracted twice more with dichloromethane. The combined organic extracts are washed with 10% sulfuric acid, saturated sodium bicarbonate solution and with brine. After drying on sodium sulfate, the solvent is spun off, and the residue is chromatographed on silica gel (mobile solvent ethyl acetate/hexane). 732.8 mg (73.76%) is isolated as a mixture that consists of 4-(4-fluoro-2-methoxyphenyl)-2-hydroxy-2-(trifluoromethyl)hexanal and 4-(4-fluoro-2-methoxyphenyl)-2-hydroxy-3-methyl-2-(trifluoromethyl)pentanal.

500 mg (1.62 mmol) of the mixture that consists of 4-(4-fluoro-2-methoxyphenyl)-2-hydroxy-2-(trifluoromethyl)hexanal and 4-(4-fluoro-2-methoxyphenyl)-2-hydroxy-3-methyl-2-(trifluoromethyl)pentanal are mixed in 10 ml of glacial acetic acid with 259.79 mg (1.62 mmol) of 5-amino-isoquinolin-1(2H)-one and stirred for three days at room temperature. The reaction mixture is spun until dry, and the residue is drawn off three times with toluene and twice with dichloromethane. After the residue is chromatographed on a Flashmaster, 322.9 mg (44.2%) of the mixture of the two imines is obtained. 322.9 g (0.72 mmol) of the mixture, described in the preceding section, that consists of 5-{[4-(4-fluoro-2-methoxyphenyl)-2-hydroxy-2-(trifluoromethyl)hexylidene]amino}isoquinolin-1(2H)-one and 5-{[4-(4-fluoro-2-methoxyphenyl)-2-hydroxy-3-methyl-2-(trifluoromethyl)pentylidene]amino}isoquinolin-1(2H)-one is introduced into 3.2 ml of dichloromethane and mixed at 0° C. drop by drop with 7.2 ml of a 1 M solution of boron tribromide in dichloromethane. After four hours of stirring at 0 to 5° C., the reaction mixture is carefully added to a mixture that consists of ice and saturated sodium bicarbonate solution. After three cycles of extraction with ethyl acetate, the combined extracts are washed with brine, dried on sodium sulfate, and the solvent is spun off. After complicated silica-gel chromatography, 2.8 mg (1.8%) of the desired compound is obtained.

¹H-NMR (400 MHz, CD₃OD): δ=1.18 (3H), 1.49 (3H), 2.37 (1H), 3.40 (1H), 5.02 (1H), 6.40 (2H), 6.78 (1H), 7.03 (1H), 7.12 (1H), 7.49 (1H), 7.69 (1H).

### EXAMPLE 20

### 5-[(7,8-Difluoro-2-methylquinazolin-5-yl)amino]-3-fluoro-5,6,7,8-tetrahydro-7,8-dimethyl-6-(trifluoromethyl)naphthalene-1,6-diol

226.5 mg (0.46 mmol) of a mixture that consists of 1-[(7,8-difluoro-2-methylquinazolin-5-yl)imino]-4-(4-fluoro-2-methoxyphenyl)-2-(trifluoromethyl)hexan-2-ol and 1-[(7,8-difluoro-2-methylquinazolin-5-yl)imino]-4-(4-fluoro-2-methoxyphenyl)-3-methyl-2-(trifluoromethyl)pentan-2-ol is cyclized as usual with the aid of boron tribromide (reaction temperature 40 to −10° C). After chromatography, 19.4 mg (17.6%) of the desired product is isolated.

¹H-NMR (400 MHz, CD₃OD): δ=0.77 (3H), 1.43 (3H), 2.25 (1H), 3.92 (1H), 5.30 (1H), 6.81 (1H), 6.89-6.99 (1H), 7.24 (1H), 9.55 (1H).

### EXAMPLE 21

### 5-{[7-Fluoro-2,5-dihydroxy-3,4-dimethyl 2-(trifluoromethyl)-1,2,3,4-tetrahydronaphthalen-1-yl]amino}isochromen-1-one

366.1 mg (0.42 mmol) of a mixture that consists of 5-{[4-(4-fluoro-2-methoxyphenyl)-2-hydroxy-2-(trifluoromethyl)hexylidene]amino}isochromen-1-one and 5-{[4-(4-fluoro-2-methoxyphenyl)-2-hydroxy-3-methyl-2-(trifluoromethyl)pentylidene]-amino}-isochromen-1-one is cyclized as usual with the aid of boron tribromide. After complicated chromatography, 6.8 mg (1.9%) of the desired product is ultimately isolated.

¹H-NMR (400 MHz, CD₃OD): δ=1.18 (3H), 1.49 (3H), 2.37 (1H), 3.40 (1H), 5.02 (1H), 6.38-6.48 (2H), 6.80 (1H), 7.18 (1H), 7.35 (1H), 7.43 (1H), 7.60 (1H).

### EXAMPLE 22

### 5-{[7-Fluoro-2,5-dihydroxy-3,4-dimethyl 2-(trifluoromethyl)-1,2,3,4-tetrahydronaphthalen-1-yl]amino}quinolin-2(1H)-one

314.4 mg (0.7 mmol) of a mixture that consists of 5-{[4-(4-fluoro-2-methoxyphenyl)-2-hydroxy-2-(trifluoromethyl)hexylidene[amino}quinolin-2(1H)-one and 5-{[4-(4-fluoro-2-methoxyphenyl)-2-hydroxy-3-methyl-2-(trifluoromethyl)-pentylidene]amino}-quinolin-2(1H)-one is cyclized as described with boron tribromide. After chromatography, 2 mg (1.31%) of the desired compound is obtained.

¹H-NMR (300 MHz, CD₃OD): δ=0.59 (3H), 1.34 (3H), 2.08 (1H), 3.88 (1H), 4.95 (1H), 6.51 (1H), 6.69 (1H), 6.73-6.92 (2H), 7.12 (1H), 7.32 (1H), 8.12 (1H).

### EXAMPLE 23

### 5-{[7-Fluoro-2,5-dihydroxy-3,4-dimethyl-2-(trifluoromethyl)-1,2,3,4-tetrahydronaphthalen-1-yl]amino}-2,3-dihydroisoindol-1-one

301 mg (0.68 mmol) of a mixture that consists of 5-{[4-(4-fluoro-2-methoxyphenyl)-2-hydroxy-2-(trifluoromethyl)hexylidene]amino}-2,3-dihydroisoindol-1-one and 5-{[4-(4-fluoro-2-methoxyphenyl)-2-hydroxy-3-methyl-2-(trifluoromethyl)pentylidene]amino}-2,3-dihydroisoindol-1-one is cyclized as described with boron tribromide. After chromatography, 5.4 mg (3.71%) of the desired compound is obtained.

¹H-NMR (400 MHz, CD₃OD): δ=1.12 (3H), 1.40 (3H), 2.38 (1H), 3.40 (1H), 4.20-4.34 (2H), 5.01 (1H), 6.38-6.49 (2H), 6.99 (1H), 7.15 (1H), 7.38 (1H).

### EXAMPLE 24

### 2,3-Difluoro-7,8-dimethyl-5-[(2-methylquinazolin-5-yl)amino]-6-(trifluoromethyl)-5,6,7,8-tetrahydronaphthalene-1,6-diol

### 4-(3,4-Difluoro-2-methoxyphenyl)-2-hydroxy-3-methyl-2-(trifluoromethyl)pentanal

Analogously to Example 1, 1,2-difluoro-3-methoxy-4-(1-methylpropenyl)benzene can be produced as an E/Z mixture from 2,3-difluorophenol.

747 mg (820 μmol) of [Cu(S,S)-2,2-bis(4,5-dihydro-4-phenyloxazolin-2-yl)propane)(H₂O)₂] ((SbF₆)₂, in 40 ml of dichloromethane, is added in drops at 0° C. over 30 minutes to 3.27 g (16.5 mmol) of 2,3-difluoro-6-(1-methylpropenyl)anisole, 4.35 ml (33 mmol) of ethyltrifluoropyruvate and 3.5 g of molecular sieve. The reaction mixture is stirred for 16 hours at room temperature, and the reaction mixture is purified by means of column chromatography on silica gel (hexane/ethyl acetate 10-20%). 3.03 g of 4-(3,4-difluoro-2-methoxyphenyl)-2-hydroxy-3-methyl-2-(trifluoromethyl)pent-4-enoic acid ethyl ester is obtained as a mixture of diastereomers. 3.03 g (45.3 mmol) of 4-(3,4-difluoro-2-methoxyphenyl)-2-hydroxy-3-methyl-2-(trifluoromethyl)pent-4-enoic acid ethyl ester is cooled in 85 ml of diethyl ether to −20° C., and 625 mg (16.5 mmol) of lithium aluminum hydride is added in portions in solid form over 10 minutes. It is stirred for 2 hours at 0° C. and poured into saturated ammonium chloride solution. The suspension is filtered through Celite and thoroughly rewashed with ethyl acetate. The phases of the filtrate are separated, and it is extracted again with ethyl acetate. It is washed with saturated sodium chloride solution, dried on sodium sulfate, the solvent is removed in a vacuum, and after column chromatography on silica gel (hexane/ethyl acetate 10-50%), 0.48 g of 4-(3,4-difluoro-2-methoxy-phenyl)-3-methyl-2-(trifluoromethyl)pent-4-ene-1,2-diol and 2.1 g of the corresponding aldehyde are obtained. 0.95 g (2.9 mmol) of 4-(3,4-difluoro-2-methoxyphenyl)-3-methyl-2-(trifluoromethyl)pent-4-ene-1,2-diol is dissolved in 100 ml of ethanol, and 100 mg of palladium on carbon (10%) is added. The suspension is stirred under a hydrogen atmosphere at 100 bar until the reaction is completed. The mixture is filtered through Celite and rewashed thoroughly with ethanol. After the solvent is removed, and after column chromatography on silica gel (hexane/ethyl acetate 30-60%), 0.62 g of 4-(3,4-difluoro-2-methoxyphenyl)-3-methyl-2-(trifluoromethyl)-pentane-1,2-diol is obtained as a mixture of diastereomers. 2.55 ml (18.4 mmol) of triethylamine and, in portions over 10 minutes, 1.17 g (7.4 mmol) of pyridine SO₃ complex are added to 1.2 g (3.7 mmol) of 4-(3,4-difluoro-2-methoxyphenyl)-3-methyl-2-(trifluoromethyl)pentane-1,2-diol in 30 ml of dichloromethane and 7.4 ml of DMSO. It is stirred over 16 hours, and a mixture that consists of saturated ammonium chloride solution and tartaric acid solution is added. The mixture is stirred for another 20 minutes, the phases are separated, and it is extracted with diethyl ether. It is washed with water and dried on sodium sulfate. The solvent is removed in a vacuum and after column chromatography on silica gel (hexane/diisopropyl ether 20-60%). 608 mg is obtained as a mixture of 3 or 4 possible stereoisomers of 4-(3,4-difluoro-2-methoxyphenyl)-2-hydroxy-3-methyl-2-(trifluoromethyl)pentanal.

Stereoisomer 1: ¹H-NMR (300 MHz, CDCl₃); δ=1.04 (d, 3H), 1.17 (d, 3H), 2.54 (dq, 1H), 2.92 (dq, 1H), 3.82 (s, 1H), 3.97 (s, 3H), 6.84 (m, 2H), 9.69 (s, 1H).

Stereoisomer 2: ¹H-NMR (300 MHz, CDCl₃); δ=0.97 (d, 3H), 1.34 (d, 3H), 2.30 (dq, 1H), 3.54 (dd, 1H), 3.97 (s, 3H), 4.59 (s, 1H), 6.78 (m, 2H), 9.50 (s, 1H).

Stereoisomer 3: ¹H-NMR (300 MHz, CDCl₃); δ=1.03 (d, 3H), 1.22 (d, 3H), 2.65 (dq, 1H), 3.07 (dq, 1H), 3.97 (s, 3H), 4.19 (s, 1H), 6.78-6.86 (m, 2H), 9.63 (s, 1H).

140 mg (0.43 mmol) of 4-(3,4-difluoro-2-methoxyphenyl)-2-hydroxy-3-methyl-2-(trifluoromethyl)pentanal and 72 mg (0.45 mmol) of 5-amino 2-methylquinazoline are dissolved in 1 ml of toluene, and 0.33 ml (0.86 mmol) of titanium tert-butylate is added. The reaction mixture is heated for 2 hours to 100°, poured into water, after cooling, and stirred vigorously. The suspension is filtered through Celite and thoroughly rewashed with ethyl acetate. The phases of the filtrate are separated, and it is extracted again with ethyl acetate. It is washed with saturated sodium chloride solution, dried on sodium sulfate, the solvent is removed in a vacuum, and 181 mg of 4-(3,4-difluoro-2-methoxyphenyl)-3-methyl-1-[(2-methylquinazolin-5-yl)imino]-2-(trifluoromethyl)-pentan-2-ol is obtained as a crude product. The crude imine is dissolved in 8 ml of CH₂Cl₂ and cooled to −45° C. 0.66 ml (0.66 mmol) of a 1 M BBr₃ solution in dichloromethane is slowly added in drops over 5 minutes, and it is allowed to heat for 2 hours to 0° C. The reaction solution is poured into a mixture that consists of saturated NaHCO₃ solution and ice. It is extracted several times with ethyl acetate, washed with saturated NaCl solution and dried on Na₂SO₄. Purification by column chromatography on silica gel (hexane/ethyl acetate 30-100%) yields 64 mg of the desired product as two separate diastereomers.

Diastereomer 1: ¹H-NMR (300 MHz, DMSO [d6]); δ=1.15 (d, 3H), 1.36 (d, 3H), 2.15 (dq, 1H), 2.73 (s, 3H), 2.89 (dq, 1H), 5.21 (d, 1H), 6.02 (s, 1H), 6.66 (dd, 1H), 6.72 (d, 1H), 6.94 (d, 1H), 7.10 (d, 1H), 7.66 (t, 1H), 9.70 (s, 1H), 10.19 (s, 1H).

Diastereomer 2: ¹H-NMR (300 MHz, CD₃OD); δ=1.28 (d, 3H), 1.41 (d, 3H), 2.37 (dq, 1H), 2.75 (s, 3H), 3.12 (dq, 1H), 5.21 (s, 1H), 6.60 (dd, 1H), 6.95 (d, 1H), 7.14 (d, 1H), 7.75 (t, 1H), 9.56 (s, 1H).

### EXAMPLE 24A/24B

### (5α,6α,7α,8β)-2,3-Difluoro-5,6,7,8-tetrahydro-7,8-dimethyl-5-[(2-methylquinazolin-5-yl)amino]-6-(trifluoromethyl)naphthalene-1,6-diol (diastereomer 1) is Cleaved by means of Preparative Chiral HPLC (Chiralpak AD-H 5μ) into the Enantiomer-Pure Compounds

Enantiomer 1: Analytical HPLC: Rₜ=4.88 minutes (Chiralpak AD-H 5μ, 250×4.6 mm, hexane/ethanol 5=>50% in 20 minutes, mil/minute of flow)

Enantiomer 2: Analytical HPLC: Rₜ=7.62 minutes (Chiralpak AD-H 5μ, 250×4.6 mm, hexane/ethanol 5=>50% in 20 minutes, 1 ml/minute of flow)

### EXAMPLE 25

### 5-{[5,6-Difluoro-1,2,3,4-tetrahydro-3,4-dimethyl-2,5-dihydroxy-6-(trifluoromethyl)naphthalen-1-yl]amino}quinolin-2(1H)-one

Analogously to Example 25, 164 mg (0.43 mmol) of 4-(3,4-difluoro-2-methoxy-phenyl)-2-hydroxy-3-methyl-2-(trifluoromethyl)pentanal and 72 mg (0.45 mmol) of 5-aminoquinolin-2(1H)-one are condensed to form the corresponding imine, which then can be cyclized analogously to Example 25 with boron tribromide to form 2 diastereomers.

Diastereomer 1: ¹H-NMR (300 MHz, CD₃OD); δ=1.15 (d, 3H), 1.34 (d, 3H), 2.24 (dq, 1H), 3.10 (dq, 1H), 4.94 (d, 1H), 5.96 (d, 1H), 6.27 (d, 1H), 6.52 (d, 1H), 6.54 (dd, 1H), 6.67 (d, 1H), 7.30 (t, 1H), 8.21 (d, 1H).

Diastereomer 2: ¹H-NMR (300 MHz, CD₃OD); δ=1.28 (d, 3H), 1.39 (d, 3H), 2.33 (dq, 1H), 3.12 (dq, 1H), 5.05 (s, 1H), 6.46 (d, 1H), 6.59 (dd, 1H), 6.65 (d, 1H), 6.67 (d, 1H), 7.36 (t, 1H), 8.15 (d, 1H).

Similarly, There can be Produced:

### 5-[(7,8-Difluoro-2-methylquinazolin-5-yl)amino]-2,3-difluoro-5,6,7,8-tetrahydro 7,8-dimethyl-6-(trifluoromethyl)naphthalene-1,6-diol

### 2,3-Difluoro-5,6,7,8-tetrahydro-7,8-dimethyl-5-[(2-methylquinolin-5-ylamino]-6-(trifluoromethyl)naphthalene-1,6-diol

### 5-{[2,3-Difluoro-1,6-dihydroxy-7,8-dimethyl-6-(trifluoromethyl)-5,6,7,8-tetrahydronaphthalen-5-yl]amino}isoquinolin-1(2H)-one

### EXAMPLE 26

### 5-{[7-Chloro-6-fluoro-2,5-dihydroxy-3,4-dimethyl-2-(trifluoromethyl)-1,2,3,4-tetrahydronaphthalen-1-yl]amino}isoquinolin-1(2H)-one

### 4-(4-Chloro-3-fluoro-2-methoxyphenyl)-2-hydroxy-3-methyl-2-(trifluoromethyl)-pentanal

3.69 g (5.96 mmol) of ytterbium(III) trifluoromethanesulfonate is introduced into 132 ml of dichloromethane. After dropwise addition of 20.28 g (119.25 mmol) of ethyltrifluoropyruvate, 12.38 g (59.63 mmol) of a mixture that consists of 4-chloro-3-fluoro-2-methoxy-1-(1-methylene-propyl)benzene and 4-chloro-3-fluoro-2-methoxy-1-(1-methyl-prop-1-ene)benzene, dissolved in 33 ml of dichloromethane, is added in drops, and the batch is then stirred for four days at room temperature. For working-up, the reaction mixture is mixed with 28 ml of water, and the organic phase is separated. After drying on sodium sulfate, the solvent is spun off, and the residue is chromatographed on silica gel (mobile solvent ethyl acetate/hexane). Altogether, 13.54 g (59.1%) of a mixture that consists of (E/Z)-ethyl-4-(4-chloro-3-fluoro-2-methoxyphenyl)-2-hydroxy-2-(trifluoromethyl)-hex-4-enoate and ethyl-4-(4-chloro-3-fluoro-2-methoxyphenyl)-2-hydroxy-3-methyl-2-(trifluoromethyl)-pent-4-enoate is obtained, which contains different ratios of the two compounds in various fractions. 5.63 g (14.64 mmol) of a portion of the above-described mixture of the two esters is dissolved in 53.4 ml of diethyl ether and mixed at −5° C. in portions with 416.5 mg (10.97 mmol) of lithium aluminum hydride, and then it is stirred for three hours between −5° C. and 0° C. The reaction mixture is mixed drop by drop at 0° C. with 11 ml of saturated sodium bicarbonate solution. After the cold bath is removed, the batch is stirred vigorously at room temperature for two hours. After the batch is filtered through a glass fiber filter and after it is rewashed with diethyl ether, the solvent is spun off. The remaining residue is chromatographed on silica gel (mobile solvent ethyl acetate/hexane). 877.6 mg (35.2%) of 4-(4-chloro-3-fluoro-2-methoxyphenyl)-2-hydroxy-3-methyl-2-(trifluoromethyl)-pent-4-enal is isolated.

¹H-NMR (400 MHz, CDCl₃): δ=1.44 (3H), 3.47 (1H), 3.92 (3H), 4.00 (1H), 5.21 (1H), 5.44 (1H), 6.68 (1H), 6.99 (1H), 9.45 (1H).

700 mg (2.05 mmol) of the previously described aldehyde in 70 ml of ethanol is added to the previously hydrogenated catalyst (platinum dioxide, 140 mg, 0.62 mmol). After 330 ml of hydrogen is taken up, and after a reaction time of 2½ hours, the reaction mixture is added via a glass fiber filter. After the filter residue is rewashed with ethanol, the solvent is spun off. 422 mg (59.9%) of the desired product (as a diastereomer mixture), which is further incorporated in crude form, remains. MS (Cl) m/e (relative intensity): 360 (100%).

170 mg (0.5 mmol) of 4-(4-chloro-3-fluoro-2-methoxyphenyl)-2-hydroxy-3-methyl-2-(trifluoromethyl)-pentanal is mixed in 2.12 ml of glacial acetic acid with 79.5 mg (0.5 mmol) of 5-amino-isoquinolin-1(2H)-one, and it is stirred for four days at room temperature. The reaction mixture is spun until dry, and the residue is drawn off three times with toluene. After the residue is chromatographed on silica gel (mobile solvent ethyl acetate/hexane), 150.9 mg (62.7%) of the desired imine is obtained. MS (Cl) m/e (relative intensity): 485 (20%).

### 5-{[7-Chloro-6-fluoro-2,5-dihydroxy-3,4-dimethyl-2-(trifluoromethyl)-1,2,3,4-tetrahydronaphthalen-1-yl]amino}-isoquinolin-1(2H)-one, nonpolar diastereomer and 5-{[7-chloro-6-fluoro-2,5-dihydroxy-3,4-dimethyl-2-(trifluoromethyl)-1,2,3,4-tetrahydronaphthalen-1-yl]amino}-isoquinolin-1(2H)-one, Polar Diastereomer

200 mg (0.45 mmol) of the previously described imine is dissolved in dichloromethane and mixed at 0° C. with 4.5 ml of a one-molar boron tribromide solution in dichloromethane. After 3½ hours of stirring at ice bath temperature, the reaction mixture is poured into a mixture that consists of ice and saturated sodium bicarbonate solution. After dilution with ethyl acetate, the mixture is stirred vigorously for two hours, and the organic phase is separated. The aqueous phase is extracted again with ethyl acetate, and the combined organic extracts are then washed with water and brine. After drying on sodium sulfate, the solvent is spun off, and the residue is chromatographed on a Flashmaster (amine phase, mobile solvent methanol/dichloromethane). 13.3 mg (6.23%) of the nonpolar diastereomer and 3.4 mg (1.59%) of the polar diastereomer of 5-{[7-chloro-6-fluoro-2,5-dihydroxy-3,4-dimethyl-2-(trifluoromethyl)-1,2,3,4-tetrahydronaphthalen-1-yl]amino}-isoquinolin-1(2H)-one are obtained.

Nonpolar diastereomer: ¹H-NMR (400 MHz, CD₃OD); δ=1.18 (3H), 1.39 (3H), 2.28 (1H), 3.13 (1H), 4.95 (1H), 6.73 (2H), 6.88 (1H), 7.19 (1H), 7.32 (1H), 7.68 (1H).

Polar diastereomer: ¹H-NMR (400 MHz, CD₃OD); δ=1.29 (3H), 1.42 (3H), 2.36 (1H), 3.16 (1H), 5.08 (1H), 6.74 (1H) 6.80 (1H), 7.00 (1H), 7.12 (1H), 7.39 (1H), 7.69 (1H).

### EXAMPLE 27

### 5-{[7-Chloro-6-fluoro-2,5-dihydroxy-3,4-dimethyl-2-(trifluoromethyl)-1,2,3,4-tetrahydronaphthalen-1-yl]amino}1,3-dihydro-2H-indol-2-one

195 mg (0.41 mmol) of 5-{[4-(4-chloro-3-fluoro-2-methoxyphenyl)-2-hydroxy-3-methyl-2-(trifluoromethyl)pentylidene]amino}-1,3-dihydro-2H-indol-2-one is cyclized as described. 10.1 mg (5.34%) of the nonpolar diastereomer and 4.7 mg (2.48%) of the polar diastereomer of 5-{[7-chloro-6-fluoro-2,5-dihydroxy-3,4-dimethyl-2-(trifluoromethyl)-1,2,3,4-tetrahydronaphthalen-1-yl]amino}-1,3-dihydro-2H-indol-2-one are isolated.

Nonpolar diastereomer: ¹H-NMR (400 MHz, CD₃OD); δ=1.18 (3H), 1.35 (3H), 2.20 (1H), 3.09 (1H), 3.20-3.48 (2H), 4.90 (1H), 6.19 (1H), 6.31 (1H), 6.81 (1H), 7.02 (1H).

Polar diastereomer: ¹H-NMR (400 MHz, CD₃OD); δ=1.28 (3H), 1.40 (3H), 2.29 (1H), 3.12 (1H), 3.28-3.38 (2H), 4.93 (1H), 6.34 (1H), 6.50 (1H), 6.83 (1H), 7.09 (1H).

### EXAMPLE 28

### 5-{[7-Chloro-6-fluoro-2,5-dihydroxy-3,4-dimethyl-2-(trifluoromethyl)-1,2,3,4-tetrahydronaphthalen-1-yl]amino}isochromen-1-one

371.9 mg (0.76 mmol) of a mixture that consists of 5-{[4-(4-chloro-3-fluoro-2-methoxyphenyl)-2-hydroxy-3-methyl-2-(trifluoromethyl)pentylidene]amino}-isochromen-1-one and 5-{[4-(4-chloro-3-fluoro-2-methoxyphenyl)-2-hydroxy-2-(trifluoromethyl)hexylidene]amino}-isochromen-1-one is cyclized with boron tribromide as already described several times. 1.5 mg (0.83%) of the desired compound is isolated.

¹H-NMR (400 MHz, CD₃OD): δ=1.13 (3H), 1.43 (3H), 2.39 (1H), 3.48 (1H), 5.02 (1H), 6.74 (1H), 6.80 (1H), 7.20 (1H), 7.38 (1H), 7.43 (1H), 7.60 (1H).

### EXAMPLE 29

### 5-{[7-Chloro-6-fluoro-2,5-dihydroxy-3,4-dimethyl-2-(trifluoromethyl)-1,2,3,4-tetrahydronaphthalen-1-yl]amino}quinolin-2(1H)-one

264.3 mg (0.54 mmol) of a mixture that consists of 5-{[4-(4-chloro-3-fluoro-2-methoxyphenyl)-2-hydroxy-3-methyl-2-(trifluoromethyl)pentylidene]amino}-isoquinolin-1(2H)one and 5-{[4-(4-chloro-3-fluoro-2-methoxyphenyl)-2-hydroxy-2-(trifluoromethyl)hexylidene]amino}isoquinolin-2(1H)-one is cyclized with the aid of boron tribromide. 7.4 mg (5.8%) of the desired compound is isolated.

¹H-NMR (400 MHz, CD₃OD): δ=1.13 (3H), 1.43 (3H), 2.39 (1H), 3.49 (1H), 5.04 (1H), 6.49 (1H), 6.63 (1H), 6.70 (1H), 6.78 (1H), 7.39 (1H), 8.15 (1H).

### In a Similar Way to the Preceding Instructions, in Addition There can be Produced

a) 5-{[7-Chloro-2,5-dihydroxy-3,4,6-trimethyl-2-(trifluoromethyl)-1,2,3,4-tetrahydronaphthalen-1-yl]amino}isoquinolin-1(2H)-one
b) 5-{[7-Chloro-2,5-dihydroxy-3,4,6-trimethyl-2-(trifluoromethyl)-1,2,3,4-tetrahydronaphthalen-1-yl]amino{-2,3-dihydroisoindol-1-one
c) 5-{[7-Chloro-2,5-dihydroxy-3,4,6-trimethyl-2-(trifluoromethyl)-1,2,3,4-tetrahydronaphthalen-1-yl]amino}isochromen-1-one
d) 3-Chloro-2,7,8-trimethyl-5-[(2-methylquinazolin-5-yl)amino]-6-(trifluoromethyl)-5,6,7,8-tetrahydronaphthalene-1,6-diol
e) 5-{[7-Chloro-2,5-dihydroxy-3,4,6-trimethyl-2-(trifluoromethyl)-1,2,3,4-tetrahydronaphthalen-1-yl]amino}quinolin-2(1H)-one
f) 5-{[2,5-Dihydroxy-3,4-dimethyl-7-(prop-2-yl)-2-(trifluoromethyl)-1,2,3,4-tetrahydronaphthalen-1-yl]amino}isoquinolin-1(2H)-one
g) 5-{[2,5-Dihydroxy-3,4-dimethyl-7-(prop-2-yl)-2-(trifluoromethyl)-1,2,3,4-tetrahydronaphthalen-1-yl]amino}-2,3-dihydroisoindol-1-one
h) 5-{[2,5-Dihydroxy-3,4-dimethyl-7-(prop-2-yl)-2-(trifluoromethyl)-1,2,3,4-tetrahydronaphthalen-1-yl]amino}isochromen-1-one
i) 5-{[2,5-Dihydroxy-3,4-dimethyl-7-(prop-2-yl)-2-(trifluoromethyl)-1,2,3,4-tetrahydronaphthalen-1-yl]amino}quinolin-2(1H)-one
j) 5,6,7,8-Tetrahydro-5-[(2-methylquinazolin-5-yl)amino]-7,8-dimethyl-3-(prop-2-yl)-6-(trifluoromethyl)naphthalene-1,6-diol
k) 5-{[6-Fluoro-3,4-diethyl-2,5-dihydroxy-2-trifluoromethyl-1,2,3,4-tetrahydronaphthalen-1-yl]amino}quinolin-2(1H)-one
l) 2-Fluoro-5-[(2-methylquinazolin-5-yl)amino]-7,8 diethyl-5,6,7,8-tetrahydro-6-(trifluoromethyl)naphthalene-1,6-diol

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosures of all applications, patents and publications, cited herein and of corresponding German application No. 102005014089.0, filed Mar. 22, 2005 and U.S. Provisional Application Ser. No. 60/670,260, filed Apr. 12, 2005, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Stereoisomers of general formula (I), 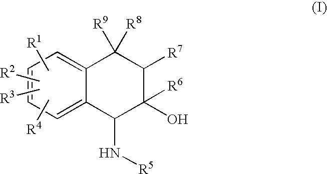
in which
R¹ and R², independently of one another, mean a hydrogen atom, a hydroxy group, a halogen atom, an optionally substituted (C₁-C₁₀)-alkyl group, an optionally substituted (C₁-C₁₀)-alkoxy group, a (C₁-C₁₀)-alkylthio group, a (C₁-C₅)-perfluoroalkyl group, a cyano group, or a nitro group, or
R¹ and R² together mean a group that is selected from the groups —O—(CH₂)ₙ—O—, —O—(CH₂)ₙ—CH₂—, —O—CH═CH—, —(CH₂)ₙ₊₂—, —NH—(CH₂)ₙ₊₁, N(C₁-C₃-alkyl)-(CH₂)ₙ₊₁, and —NH—N═CH—, whereby n=1 or 2, and the terminal atoms are linked to directly adjacent ring-carbon atoms,
or NR¹⁰R¹¹, whereby R¹⁰ and R¹¹, independently of one another, can be hydrogen, C₁-C₅-alkyl or (CO)—C₁-C₅-alkyl,
R³ and R⁴, independently of one another, mean a hydrogen atom, a hydroxy group, a halogen atom, a cyano group, an optionally substituted (C₁-C₁₀)-alkyl group, an optionally substituted (C₁-C₁₀)-alkoxy group, a (C₁-C₁₀)-alkylthio group, or a (C₁-C₅)-perfluoroalkyl group,
R⁵ means a C₁-C₁₀-alkyl group, or a C₁-C₁₀-alkyl group that is substituted by one or more groups that are selected from hydroxy groups, halogen atoms, or (C₁-C₅)-alkoxy groups,
an optionally substituted (C₃-C₇)-cycloalkyl group,
an optionally substituted heterocyclyl group,
an optionally substituted aryl group,
a monocyclic or bicyclic heteroaryl group that optionally is substituted, independently of one another, by one or more groups selected from (C₁-C₅)-alkyl groups (which optionally can be substituted by 1-3 hydroxy or 1-3 COOR¹⁰ groups), (C₁-C₅)-alkoxy groups, hydroxy groups, halogen atoms, or 1-2 (C₁-C₃)-exoalkylidene groups or that optionally contains 1-4 nitrogen atoms and/or 1-2 oxygen atoms and/or 1-2 sulfur atoms and/or 1-2 keto groups, whereby this group can be linked via any position to the amine of the tetrahydronaphthalene system and optionally can be hydrogenated at one or more sites,
R⁶ means a (C₁-C₅)-alkyl group or an optionally partially or completely fluorinated (C₁-C₅)-alkyl group, a (C₃-C₇)-cycloalkyl group, a (C₃-C₇)-cycloalkyl(C₁-C₈)alkyl group, a (C₃-C₇)-cycloalkyl(C₂-C₈)alkenyl group, a heterocyclyl group, a heterocyclyl(C₁-C₈)alkyl group, a heterocyclyl(C₂-C₈)alkenyl group, an aryl group, an aryl(C₁-C₈)alkyl group, an aryl(C₂-C₈)alkenyl group, an aryl(C₂-C₈)alkinyl group; a monocyclic or bicyclic heteroaryl group that optionally is substituted by one or more keto groups, (C₁-C₅)-alkyl groups, (C₁-C₅)-alkoxy groups, halogen atoms, or (C₁-C₃)-exoalkylidene groups and that contains one or more nitrogen atoms and/or oxygen atoms and/or sulfur atoms,
a heteroaryl(C₁-C₈)alkyl group or a heteroaryl(C₂-C₈)alkenyl group,
whereby these groups can be linked via any position to the tetrahydronaphthalene system and optionally can be hydrogenated at one or more sites,
R⁷ means a halogen atom, or a (C₁-C₁₀)-alkyl group, which optionally can be substituted by OR¹⁰, SR¹⁰, N(R¹⁰R¹¹) or 1-3 halogen atoms,
R⁸ and R⁹, independently of one another, mean a hydrogen atom, a halogen atom, a (C₁-C₅)alkyl group, which can be substituted with OR¹⁰, SR¹⁰, or N(R¹⁰R¹¹), a cyano group, or, together with the carbon atom of the ring system, a (C₃-C₆)-cycloalkyl ring, or together a (C₁-C₅)-alkylidene group that optionally is substituted by hydroxy, halogen or cyano, or
R⁷ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms.

2. Stereoisomers of general formula (I), 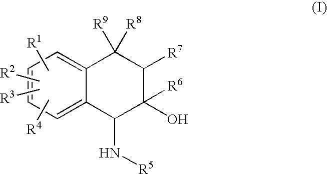
in which
R¹ and R², independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkythio group, a (C₁-C₅)-perfluoroalkyl group, a cyano group, or a nitro group, or
R¹ and R² together mean a group that is selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms, or NR¹⁰R¹¹, whereby R¹⁰ and R¹¹, independently of one another, can be hydrogen, C₁-C₅-alkyl or (CO)—C₁-C₅-alkyl,
R³ and R⁴, independently of one another, mean a hydrogen atom, a hydroxy group, a halogen atom, an optionally substituted (C₁-C₁₀)-alkyl group, a (C₁-C₁₀)-alkoxy group, a (C₁-C₁₀)-alkylthio group, a (C₁-C₅)-perfluoroalkyl group, or a cyano group,
R⁵ means a C₁-C₁₀-alkyl group, a C₁-C₁₀-alkyl group that is substituted by one or more groups selected from 1-3 hydroxy groups, halogen atoms, or 1-3 (C₁-C₅)-alkoxy groups, an optionally substituted phenyl group, a monocyclic or bicyclic heteroaryl group that optionally is substituted by 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups and that contains 1-4 nitrogen atoms and/or 1-2 oxygen atoms and/or 1-2 sulfur atoms and/or 1-2 keto groups, whereby these groups can be linked via any position to the amine of the tetrahydronaphthalene system and optionally can be hydrogenated at one or more sites,
R⁶ means a (C₁-C₅)-alkyl group or an optionally partially or completely fluorinated (C₁-C₅)-alkyl group, an aryl group, an aryl(C₁-C₈)alkyl group, an aryl(C₂-C₈)alkenyl group, a (C₃-C₇)cycloalkyl group, a (C₃-C₇)cycloalkyl(C₁-C₈)alkyl group, or a (C₃-C₇)cycloalkyl(C₂-C₈)alkenyl group,
R⁷ means a halogen atom, a (C₁-C₁₀)-alkyl group, which optionally can be substituted with OR¹⁰, SR¹⁰, N(R¹⁰R¹¹) or 1-3 halogen atoms,
R⁸ and R⁹, independently of one another, mean a hydrogen atom, a halogen atom, a methyl or ethyl group, which should be substituted with OR¹⁰, SR¹⁰, or NR¹⁰R¹¹, a cyano group, or, together with the carbon atom of the tetrahydronaphthalene ring, a (C₃-C₆)-cycloalkyl ring, or together a (C₁-C₅)-alkylidene group, or
R⁷ and R⁸ together mean an annelated five- to eight-membered, saturated or unsaturated carbocyclic compound or heterocyclic compound, which optionally is substituted by 1-2 keto groups, 1-2 (C₁-C₅)-alkyl groups, 1-2 (C₁-C₅)-alkoxy groups, or 1-4 halogen atoms.

3. Stereoisomers of general formula (I), 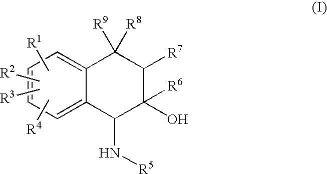
in which
R¹ and R², independently of one another, mean a hydrogen atom, a hydroxy group, a halogen atom, an optionally substituted (C₁-C₅)-alkyl group, a (C₁-C₅)-alkoxy group, or a cyano group, or
R¹ and R² together mean a group that is selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms,
R³ and R⁴, independently of one another, mean a hydrogen atom, a hydroxy group, a halogen atom, an optionally substituted (C₁-C₅)-alkyl group, or a (C₁-C₅)-alkoxy group,
R⁵ means a C₁-C₁₀-alkyl group, a C₁-C₁₀-alkyl group that is substituted by one or more groups selected from 1-3 hydroxy groups or halogen atoms; a phenyl, phthalidyl, isoindolyl, dihydroindolyl, dihydroisoindolyl, dihydroisoquinolinyl, thiophthalidyl, benzoxazinonyl, phthalazinonyl, quinolinyl, isoquinolinyl, quinolonyl, isoquinolonyl, indazolyl, benzothiazolyl, quinazolinyl, quinoxalinyl, cinnolinyl, phthalazinyl, chromenyl, isochromenyl, chromenonyl, isochromenonyl, 1,7- or 1,8-naphthyridinyl, dihydroindolonyl, dihydroisoindolonyl, benzimidazole or indolyl group that optionally is substituted by one or more groups selected from 1-2 keto groups, 1-2 (C₁-C₅)-alkyl groups, 1-2 (C₁-C₅)-alkoxy groups, 1-3 hydroxy groups, 1-3 halogen atoms, or 1-2 (C₁-C₃)-exoalkylidene groups, whereby these groups can be linked via any position to the amine of the tetrahydronaphthalene system and optionally can be hydrogenated at one or more sites,
R⁶ means a (C₁-C₅)-alkyl group or an optionally partially or completely fluorinated (C₁-C₅)-alkyl group,
R⁷ means a halogen atom, a methyl or ethyl group, which should be substituted with OR¹⁰, SR¹⁰, N(R¹⁰R¹¹) or 1-3 halogen atoms,
R⁸ and R⁹, independently of one another, mean a hydrogen atom, a halogen atom, a methyl or ethyl group, which should be substituted with OR¹⁰, SR¹⁰, or N(R¹⁰)₂, a cyano group, or, together with the carbon atom of the tetrahydronaphthalene ring, a (C₃-C₆)-cycloalkyl ring, or together a (C₁-C₅)-alkylidene group, or
R⁷ and R⁸ together mean an annelated five- to eight-membered, saturated or unsaturated carbocyclic or heterocyclic compound, which optionally is substituted by 1-2 keto groups, 1-2 (C₁-C₅)-alkyl groups, 1-2 (C₁-C₅)-alkoxy groups, or 1-4 halogen atoms.

4. Stereoisomers of general formula (I), 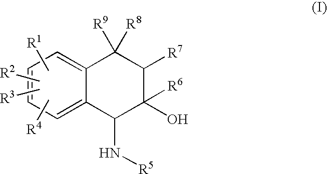
in which
R¹ and R², independently of one another, mean a hydrogen atom, a hydroxy group, a halogen atom, a (C₁-C₅)-alkyl group, a (C₁-C₅)-alkoxy group, or together a group selected from the groups —O—(CH₂)ₙ—O—, —O—(CH₂)ₙ—CH₂—, —O—CH═CH—, and —(CH₂)ₙ₊₂—, whereby n=1 or 2, and the terminal atoms are linked to directly adjacent ring-carbon atoms,
R³ means a hydrogen atom, a hydroxy group, a halogen atom, an optionally substituted (C₁-C₅)-alkyl group, or a (C₁-C₅)-alkoxy group,
R⁴ means a hydrogen atom,
R⁵ means a phenyl, phthalidyl, isoindolyl, dihydroindolyl, dihydroisoindolyl, dihydroisoquinolinyl, thiophthalidyl, benzoxazinonyl, phthalazinonyl, chromenyl, isochromenyl, chromenonyl, isochromenonyl, quinolinyl, isoquinolinyl, quinolonyl, isoquinolonyl, indazolyl, benzothiazolyl, quinazolinyl, quinoxalinyl, cinnolinyl, phthalazinyl, 1,7- or 1,8-naphthyridinyl, dihydroindolonyl, dihydroisoindolonyl, benzimidazole or indolyl group that optionally is substituted by one or more groups selected from 1-2 keto groups, 1-2 (C₁-C₅)-alkyl groups, 1-2 (C₁-C₅)-alkoxy groups, 1-3 hydroxy groups, 1-3 halogen atoms, and 1-2 (C₁-C₃)-exoalkylidene groups,
whereby these groups can be linked via any position to the amine of the tetrahydronaphthalene system and optionally can be hydrogenated at one or more sites,
R⁶ means a completely fluorinated (C₁-C₅)-alkyl group,
R⁷ means a methyl or ethyl group,
R⁸ and R⁹, independently of one another, mean a hydrogen atom,
a methyl or ethyl group, or, together a methylene or ethylidene group, or
R⁷ and R⁸ together mean an annelated five- to eight-membered, saturated or unsaturated carbocyclic compound.

5. Use of the stereoisomers according to claim 1 for the production of a pharmaceutical agent.

6. Use of the stereoisomers of claim 1 for the production of a pharmaceutical agent for treating inflammatory diseases.

7. Pharmaceutical preparations that contain at least one stereoisomer according to claim 1 or mixtures thereof as well as pharmaceutically compatible vehicles.

8. Stereoisomers of general formula I, according to claim 1, in the form of salts with physiologically compatible anions.

9. Process for the production of stereoisomers of general formula I, in which the radicals, if not otherwise mentioned, have the meanings that are defined in claim 1, characterized in that either
a)
Stereoisomers of general formula (II) 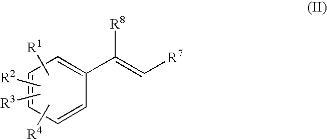
in which R¹, R², R³, R⁴, R⁷ and R⁸ have the meanings that are mentioned in claim 1, are converted by an optionally enantioselectively conducted En reaction with α-keto acids R⁶(CO)COOR¹⁰ in the presence of optionally chiral Lewis acids into compounds of general formula (III) 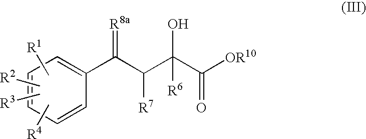
by reduction and reaction with amines of formula R⁵—NH₂, whereby R⁵ has the meaning that is indicated in claim 1,
the compounds of general formula (IV) are produced 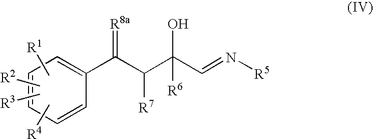
in which R¹, R², R³, R⁴, R⁵ and R⁷ have the meanings that are indicated in claim 1, and R^{8a} corresponds to the common meaning of R⁸ and R⁹ as an alkylidene group, then they are cyclized to compounds of general formula (I) either without additional reagent or by adding inorganic or organic acids or Lewis acids at temperatures of −70° C. to 80° C.,
or
b)
compounds of formula (III), produced according to a), are introduced by hydrogenation of radical R⁹═H, by methods of cyclopropanation of radical R⁹ with the common meaning of R⁸ and R⁹═CH₂—CH₂ or by hydrohalogenation of radical R⁹=halogen, and thus compounds of general formula V are produced, 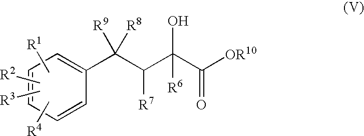
in which R¹, R², R³, R⁴, R⁶, R⁷, R⁸ and R¹⁰ have the meanings that are indicated in claim 1, by reduction and reaction with amines of formula R⁵—NH₂, whereby R⁵ has the meaning that is indicated in claim 1,
the compounds of general formula (VI) are produced, 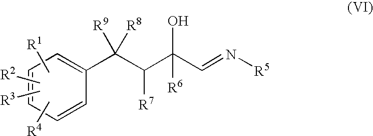
which are cyclized to compounds of general formula I either without additional reagent or by adding inorganic or organic acids or Lewis acids at temperatures of −70° C. to 80° C.

10. Process stages for the production of stereoisomers of general formula I according to claim 9, wherein stereoisomers of general formula VI, 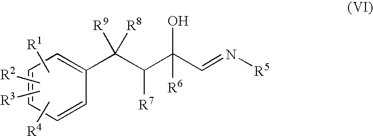
optionally are cyclized with the addition of inorganic or organic acids or Lewis acids.

11. Compounds of general formula VI according to claim 9

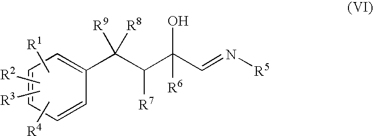

12. Process stage for the production of compounds of general formula (I), according to claim 9, wherein stereoisomers of general formula (V) 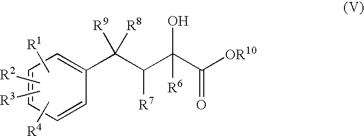
are reduced and are reacted with a corresponding amine of formula R⁵—NH₂ to form the imine of general formula (VI).

13. Compounds of formula V according to claim 9, [chemical structure]
